(19) United States
(12) Patent Application Publication
(10) Pub. No.: US 2019/0194593 A1
(43) Pub. Date: Jun. 27, 2019

(54) OBSERVATION SYSTEM, OBSERVATION APPARATUS, AND TEMPERATURE CONTROL METHOD

(71) Applicant: Olympus Corporation, Tokyo (JP)

(72) Inventors: Norihiko OZAKI, Hachioji-shi (JP); Daigo Sasaki, Hachioji-shi (JP)

(21) Appl. No.: 16/231,862

(22) Filed: Dec. 24, 2018

(30) Foreign Application Priority Data
Dec. 26, 2017 (JP) 2017-249981

(51) Int. Cl. C12M 1/00; C12M 1/36; G06K 9/00

(52) U.S. Cl. CPC C12M 41/14; G06K 9/00624; C12M 41/48

(57) ABSTRACT

According to an aspect of the invention, an observation system includes at least one observation apparatus and a thermostatic bath. The observation apparatus includes an imaging unit, a temperature sensor, and first control circuitry. The imaging unit captures an image of the sample. The temperature sensor measures an internal temperature of the observation apparatus. The first control circuitry estimates a sample temperature based on the internal temperature and calculates an indication temperature based on the sample temperature and temperature distribution data of the thermostatic chamber. The thermostatic bath includes a memory and second control circuitry. The memory stores the temperature distribution data. The second control circuitry performs the temperature control by setting a set temperature based on the indication temperature.

### CROSS-REFERENCE TO RELATED APPLICATIONS

This application is based upon and claiming the benefit of priority from prior Japanese Patent Application No. 2017-249981, filed Dec. 26, 2017, the entire contents of which are incorporated herein by reference.

### BACKGROUND OF THE INVENTION

### 1. Field of the Invention

The present invention relates to an observation system, an observation apparatus, and a temperature control method.

### 2. Description of the Related Art

In general, an imaging apparatus statically placed in an incubator with a culture vessel and imaging cultured cells or the like in the culture vessel is known in the art. For example, in culturing of cells or the like, the incubator is controlled so that the environment in the incubator is in high ambient temperature and humidity. For example, Jpn. Pat. Appln. KOKAI Publication No. 2011-110033 discloses a technique related to an incubator which controls a temperature inside a culture chamber (thermostatic chamber) by convection of gas heated by a heater or cooled by a freezer inside the culture chamber.

In the incubator in which temperature control is performed by gas that is caused to flow in the interior, there is a high possibility that a temperature difference exists in the culture chamber of the incubator. Therefore, there is a possibility that the temperature at the position of the culture vessel in the incubator will not be kept at a preset temperature of the incubator. Such temperature non-uniformity inside the incubator interferes with proper culture.

### BRIEF SUMMARY OF THE INVENTION

According to an aspect of the invention, an observation system includes at least one observation apparatus and a thermostatic bath. In the observation system, in a state in which the observation apparatus is placed in a thermostatic chamber of the thermostatic bath together with a sample to be observed, the observation apparatus and the thermostatic bath communicate with each other and temperature control in the thermostatic chamber is performed based on a set temperature. The observation apparatus includes an imaging unit, a temperature sensor, and first control circuitry. The imaging unit is configured to capture an image of the sample. The temperature sensor is configured to measure an internal temperature of the observation apparatus. The first control circuitry is configured to estimate a sample temperature, which is a temperature of the sample, based on the internal temperature and calculate an indication temperature indicated to the thermostatic bath as the set temperature based on the sample temperature and temperature distribution data acquired from the thermostatic bath through the communication. The thermostatic bath includes a memory and second control circuitry. The memory is configured to store the temperature distribution data of the thermostatic chamber. The second control circuitry is configured to perform the temperature control by setting the set temperature based on the indication temperature acquired from the observation apparatus through the communication.

According to an aspect of the invention, an observation apparatus includes an imaging unit, a temperature sensor, a memory, and control circuitry. The imaging unit is configured to capture an image of a sample to be observed. The temperature sensor is configured to measure an internal temperature of the observation apparatus. The memory is configured to store temperature distribution data of a thermostatic chamber of a thermostatic bath, in which temperature control is performed based on a set temperature in the thermostatic chamber in which the observation apparatus is placed together with the sample. The control circuitry is configured to estimate a sample temperature, which is a temperature of the sample, based on the internal temperature, calculate an indication temperature recommended as the set temperature of the thermostatic bath based on the sample temperature and the temperature distribution data, and generate display information for displaying the indication temperature.

According to an aspect of the invention, a temperature control method, which is performed in a state in which an observation apparatus is placed together with a sample in a thermostatic chamber of a thermostatic bath in which temperature control is performed based on a set temperature, the observation apparatus including an imaging unit configured to capture an image of the sample, is provided. The temperature control method includes registering temperature distribution data of the thermostatic chamber, measuring an internal temperature of the observation apparatus, estimating a sample temperature, which is a temperature of the sample, based on the internal temperature, calculating an indication temperature indicated to the thermostatic bath as the set temperature based on the sample temperature and the temperature distribution data, and setting the set temperature based on the indication temperature.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWINGS

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic view illustrating an outline of the appearance of an observation apparatus and a controller according to a first embodiment;

FIG. 2 is a schematic diagram illustrating an outline of a configuration example of an observation system according to the first embodiment;

FIG. 3 is a block diagram schematically illustrating a configuration example of the observation system according to the first embodiment;

FIG. 4 is a view for explaining a temperature distribution occurring in a thermostatic bath according to the first embodiment;

FIG. 5 is a schematic view illustrating an outline of a configuration example of an image acquisition unit and a sample according to the first embodiment;

FIG. 6 is a view for explaining temperature distribution data according to the first embodiment;

FIG. 7 is a flowchart illustrating an example of observation apparatus control processing according to the first embodiment;

FIG. 8 is a flowchart illustrating an example of observation/measurement processing according to the first embodiment;

FIG. 9 is a flowchart illustrating an example of temperature measurement/communication processing according to the first embodiment;

FIG. 10 is a flowchart illustrating an example of indication temperature calculation processing according to the first embodiment;

FIG. 11 is a view for explaining the calculation of indication temperature according to the first embodiment;

FIG. 12 is a flowchart illustrating an example of thermostatic bath control processing according to the first embodiment;

FIG. 13 is a flowchart illustrating an example of controller control processing according to the first embodiment;

FIG. 14 is a schematic view illustrating an example of display in the controller according to the first embodiment;

FIG. 15 is a schematic view illustrating an example of display on the controller according to the first embodiment;

FIG. 16 is a flowchart illustrating an example of temperature measurement/communication processing according to a second embodiment;

FIG. 17 is a flowchart illustrating an example of thermostatic bath control processing according to the second embodiment;

FIG. 18 is a flowchart illustrating an example of temperature measurement/communication processing according to a third embodiment;

FIG. 19 is a flowchart illustrating an example of thermostatic bath control processing according to the third embodiment;

FIG. 20A is a flowchart illustrating an example of controller control processing according to the third embodiment; and

FIG. 20B is a flowchart illustrating an example of controller control processing according to the third embodiment.

### DETAILED DESCRIPTION OF THE INVENTION

### First Embodiment

### <Configuration of Observation System>

### (Outline of Observation System)

The first embodiment of the present invention will be explained with reference to the drawings. An observation system 1 of this embodiment is a system which takes images of a cell, a cell group, and a tissue which are being cultured, and which makes a record of the numbers of cells or cell groups and the form thereof.

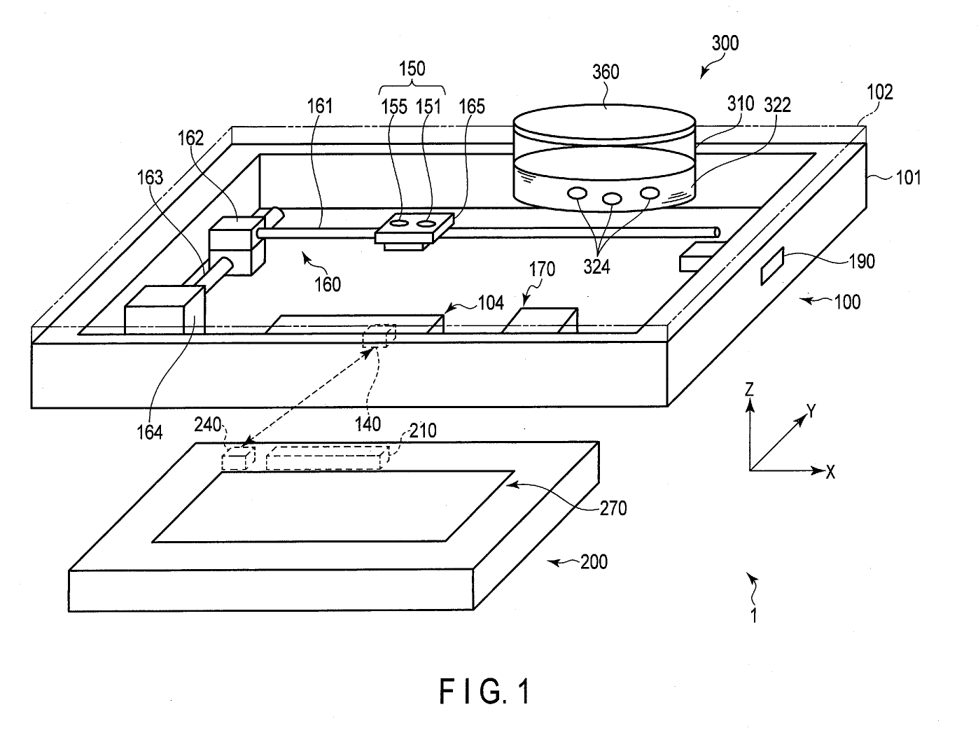

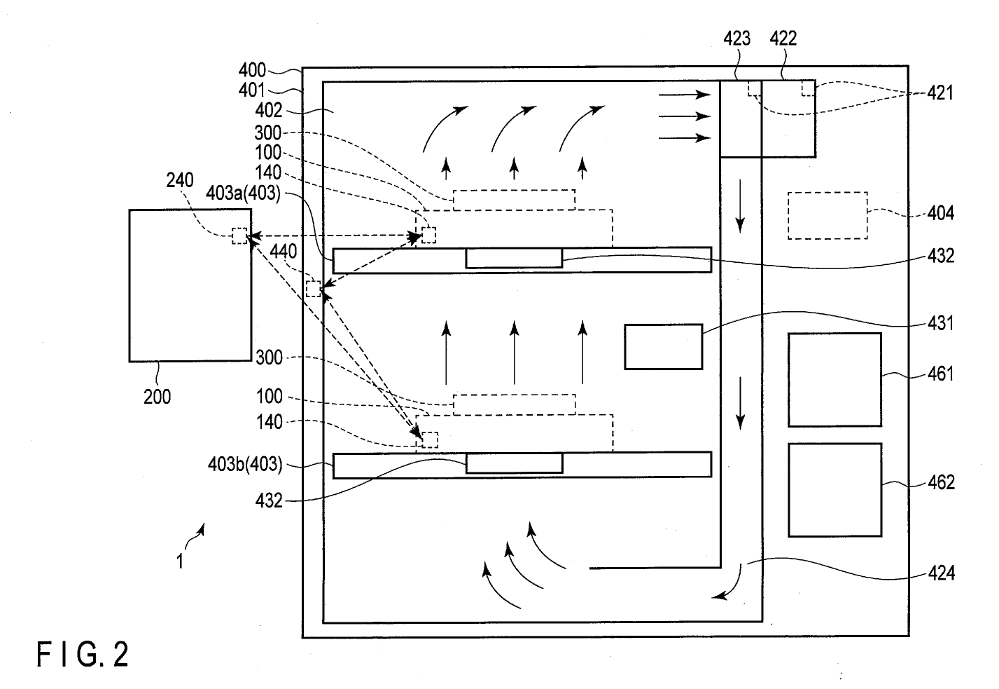

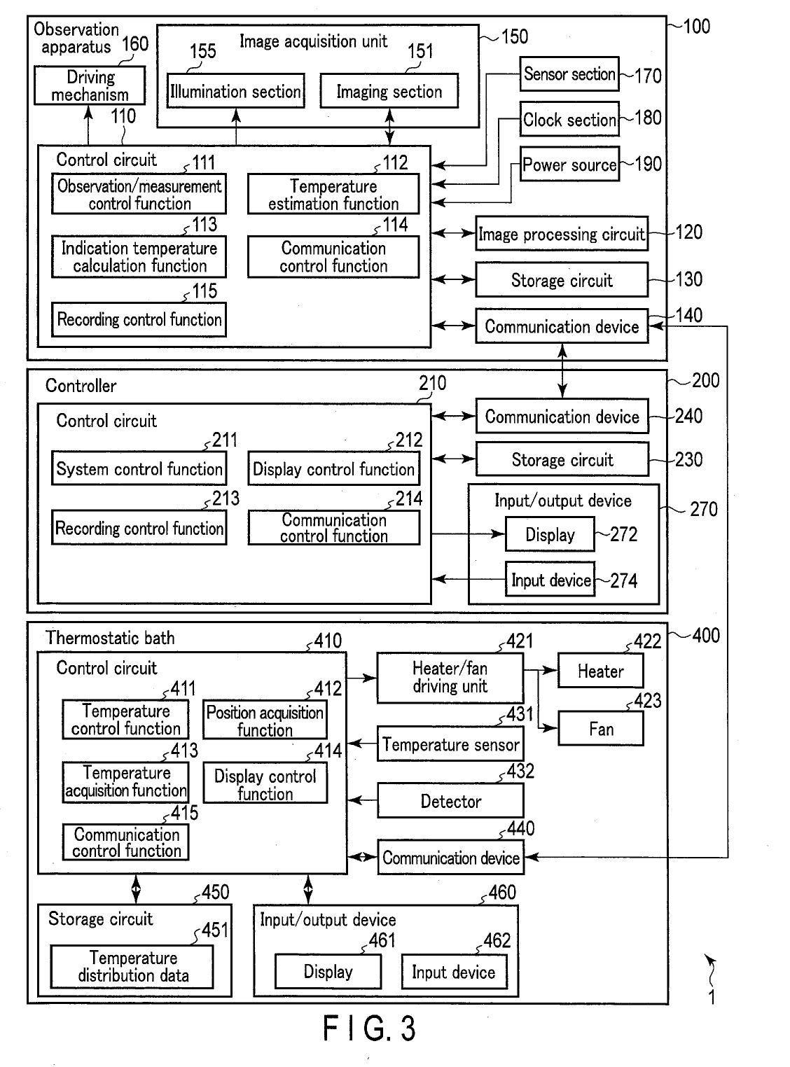

FIG. 1 is a schematic view illustrating an outline of the appearance of an observation apparatus 100 and a controller 200 provided in an observation system 1. FIGS. 2 and 3 are a schematic view and a block diagram illustrating an outline of a configuration example of the observation system 1, respectively. As illustrated in FIGS. 2 and 3, the observation system 1 of this embodiment is provided with an observation apparatus 100, a controller 200, and a thermostatic bath 400. As illustrated in FIG. 1, the observation apparatus 100 is provided with an observation side casing 101 and a transparent plate 102. The observation apparatus 100 has a roughly flat plate shape. A sample 300 to be observed is placed on top of the observation apparatus 100, that is, on the outer surface of the transparent plate 102. As illustrated in FIG. 2, the observation apparatus 100 can be placed on a shelf 403 provided in a thermostatic chamber 402 (culture chamber) of the thermostatic bath 400 while the sample 300 is placed on the top surface. As described above, the sample 300 of this embodiment can be taken in and out from the thermostatic bath 400, a clean bench, or the like while being placed on top of the observation apparatus 100. As illustrated in FIG. 2, the shelf 403 has an upper shelf 403a and a lower shelf 403b which are a plurality of shelves installed at different heights. The upper shelf 403a is installed in the upper part of the thermostatic chamber 402. The lower shelf 403b is installed in the lower part of the thermostatic chamber 402. Therefore, the observation apparatus 100 can be arranged at a different height in the thermostatic chamber 402. On the other hand, the controller 200 is installed, for example, outside the thermostatic bath 400. The observation apparatus 100 communicates with the controller 200. The controller 200 controls the operation of the observation apparatus 100. The observation apparatus 100 illuminates the sample 300 through the transparent plate 102 in a state of being placed in the thermostatic chamber 402 of the thermostatic bath 400, and captures to acquire an image of the sample 300. At this time, in the observation apparatus 100, a plurality of images can be acquired while the imaging position is changed with respect to the sample 300.

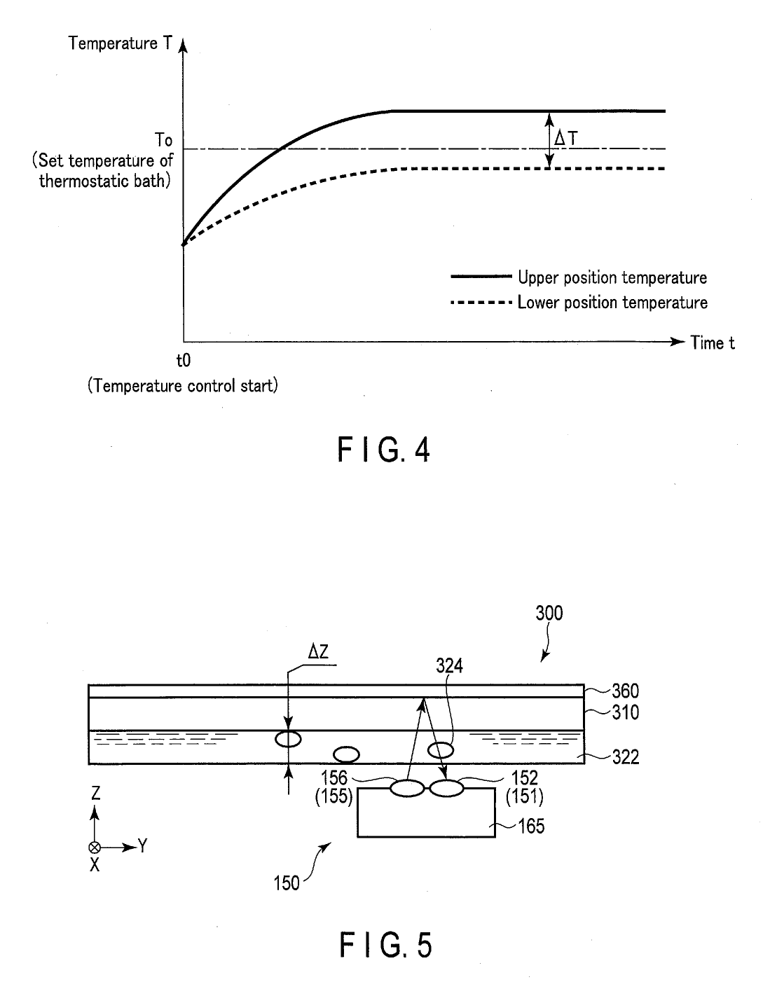

The outline of temperature control executed by the observation system 1 of this embodiment will be explained with reference to the drawings. FIG. 4 is a view for explaining a temperature distribution occurring in the thermostatic bath. In the graph illustrated in FIG. 4, a horizontal axis represents the time t from the start of the temperature control, and a vertical axis represents the temperature T inside the thermostatic bath 400. The graph illustrated in FIG. 4 shows the temporal changes of an upper position temperature and a lower position temperature. The upper position temperature is a temperature at the position (upper position) of the upper shelf 403a. The lower position temperature is a temperature at the position (lower position) of the lower shelf 403b. As illustrated in the graph in FIG. 4, when the temperature control is started in the thermostatic bath 400, the upper position temperature and the lower position temperature approaches a set temperature To of the thermostatic bath 400 set by a user. In general, during cell culture, it is important that the temperature control is performed so that the temperature of the sample 300, that is, the temperature at the position at which the observation apparatus 100 is placed is maintained within a predetermined temperature range. However, even if the temperature control reaches a steady state, a temperature distribution exists inside the thermostatic chamber 402, depending on the performance of the thermostatic bath. For example, a temperature difference ΔT exists between the upper position temperature and the lower position temperature. In addition, the position (reference control position) at which the thermostatic bath 400 measures the internal temperature of the thermostatic chamber 402 for temperature control may be different from the position at which the observation apparatus 100 is placed. As such, there is a possibility that the set temperature To and the actual temperature of the sample 300 are different according to the position at which the observation apparatus 100 is placed.

Therefore, the observation system 1 of this embodiment controls the temperature of the thermostatic bath 400 based on the indication temperature calculated by the observation apparatus 100 placed in the thermostatic bath 400 together with the sample 300 to be subjected to the temperature control. Therefore, in the observation system 1 of this embodiment, appropriate temperature control on the sample 300 can be realized regardless of the performance of the thermostatic bath 400 and the arrangement of the observation apparatus 100 in the thermostatic bath 400.

For the following explanation, X axis and Y axis orthogonal to each other are defined in a plane parallel to a plane of the observation apparatus 100 on which the sample 300 is placed, and Z axis is defined so as to be orthogonal to the X axis and the Y axis.

### (Sample)

An example of the sample 300 to be measured by the observation system 1 will be described below. The sample 300 includes, for example, a vessel 310, a culture medium 322, cells 324, and a reflecting plate 360. The culture medium 322 is in the vessel 310, and the cells 324 are cultured in the culture medium 322. The vessel 310 may be, for example, a petri dish, a culture flask, a multiwell plate, or the like. As described above, the vessel 310 is a culture vessel for culturing a biological sample, for example. The vessel 310 is not limited to any specific shape or size. The culture medium 322 may be either a liquid medium or a solid medium. The cells 324 to be measured may be either adhesive cells or floating cells. Alternatively, the cells 324 may be spheroids or tissues. In addition, the cells 324 may be derived from any organism or may be bacteria or the like. As described above, the sample 300 includes a living sample which is either the living substance itself or is derived from the living substance. The reflecting plate 360 is placed on top of the vessel 310, and reflects the illumination light incident on the sample 300 through the transparent plate 102 and illuminates the cells 324.

### (Observation Apparatus)

The transparent plate 102 of the observation apparatus 100 is made of, for example, glass. Although FIG. 1 shows that the top plate of the observation side casing 101 is entirely transparent, the observation apparatus 100 may be designed so that part of the top plate of the observation side casing 101 is a transparent plate, and the remaining part of the top plate is opaque. The term “transparent” used here means transparent to the wavelength of the illumination light. In addition, a fixing frame may be placed on the transparent plate 102 in order to unify the position at which the sample 300 is placed on the transparent plate 102 and fix the sample 300. The fixing frame may be designed so that it is arranged at a specific position with respect to the transparent plate 102. For example, the fixing frame may have the same size as the transparent plate 102. The observation apparatus 100 is in a state in which the inside thereof is hermetically sealed by a member including the observation side casing 101 and the transparent plate 102, for example.

As illustrated in FIGS. 1 to 3, the observation apparatus 100 is provided with an observation side control circuit 110, an image processing circuit 120, an observation side storage circuit 130, an observation side communication device 140, an image acquisition unit 150, a driving mechanism 160, a sensor section 170, a clock section 180, and a power source 190.

The observation side control circuit 110 controls operations of each of the elements of the observation apparatus 100. As illustrated in FIG. 3, the observation side control circuit 110 functions as an observation/measurement control function 111, a temperature estimation function 112, an indication temperature calculation function 113, a communication control function 114, and a recording control function 115. Each of the functions 111 to 115 is realized by, for example, causing the observation side control circuit 110 to execute a program recorded in the observation side storage circuit 130. The observation side control circuit 110 is an example of first control circuitry.

The observation/measurement control function 111 performs various kinds of control related to observation and measurement, such as timing and the number of times the observation and measurement are performed, and the operations of each of the elements of the observation apparatus 100 when observation and measurement are executed. The observation/measurement control function 111 functions as a position control function, an imaging control function, an illumination control function, a determination function, and a warning control function. The position control function controls the operation of the driving mechanism 160 and controls the position of the image acquisition unit 150. The imaging control function controls the operation of the imaging section 151 provided in the image acquisition unit 150 and causes the imaging section 151 to acquire the image of the sample 300. The imaging control function also performs, for example, focus adjustment, exposure adjustment, and zoom adjustment. The illumination control function controls the operation of the illumination section 155 provided in the image acquisition unit 150. The determination function makes various determinations related to the operation of the observation apparatus 100 at the time of observation and measurement. The warning control function generates a control signal for notifying or warning the user in response to the output of the determination function. The temperature estimation function 112 estimates the temperature of the sample 300 (sample temperature Tc). The estimation of the sample temperature Tc is based on, for example, the internal temperature Te of the observation apparatus 100 output from the sensor section 170. The calculation formula or the like used for estimating the sample temperature Tc is recorded in the observation side storage circuit 130. When the sensor section 170 can measure the temperature outside the observation apparatus 100, the sample temperature Tc may be calculated based on the temperature outside the observation apparatus 100. The indication temperature calculation function 113 calculates the indication temperature Tic indicated as the set temperature To in the thermostatic bath 400. The set temperature To is a target temperature at a reference control position in the temperature control of the thermostatic bath 400. The thermostatic bath 400 performs temperature control so that the temperature at the reference control position becomes the set temperature To. The calculation of the indication temperature Tic is based on, for example, the sample temperature Tc, the target temperature Tm of the position (control position) at which the sample 300 is placed, information related to the temperature distribution inside the thermostatic bath 400 (temperature distribution data of the thermostatic bath), and the set temperature To at that time. The communication control function 114 controls the operation of the observation side communication device 140 and manages the communication with the controller 200 or the thermostatic bath 400. The recording control function 115 controls the recording operation to the observation side storage circuit 130 and records the data obtained by the observation apparatus 100, the information received from the controller 200 and the thermostatic bath 400, and the like in the observation side storage circuit 130.

The image processing circuit 120 performs a variety of image processing on the image data acquired by the observation apparatus 100. The image processing circuit 120 generates a wide range of image data by combining a plurality of pieces of image data acquired at mutually different positions having the same Z position, or generates depth composite image data by combining a plurality of pieces of image data acquired at different focal lengths or Z positions. The image processing circuit 120 may generate three-dimensional image data such as a three-dimensional model of cells based on the acquired image data. The image processing circuit 120 may perform various analyses based on the obtained image. For example, the image processing circuit 120 extracts images of cells or cell groups included in the sample 300 or calculates the number of cells or cell groups based on the acquired image data. The data and the analysis result after the image processing are recorded in the observation side storage circuit 130 or transmitted to the controller 200.

For example, a program used in each of the elements of the observation apparatus 100, various parameters, a driving pattern and a scan pattern of the image acquisition unit 150, data acquired by the observation apparatus 100, and the like are recorded in the observation side storage circuit 130. The data, which is acquired by the observation apparatus 100 and recorded by the observation side storage circuit 130, includes, for example, a measurement value of measurement, a measurement start condition, an acquired image, an imaging position, an imaging condition, an analysis result, and the like. In addition, various data such as image data (pixel data), image data for recording, image data for display, and processing data at the time of operation are temporarily recorded in the observation side storage circuit 130.

The observation side communication device 140 is provided with a circuit for the observation apparatus 100 to communicate with another device. The communication between the observation apparatus 100 and the controller 200 or the thermostatic bath 400 is performed by, for example, wireless communication. The observation side communication device 140 is provided with, for example, a communication circuit compatible with Wi-Fi communication. The observation side communication device 140 may further include a communication circuit compatible with Bluetooth communication or Bluetooth Low Energy (BLE) communication. In this case, the Wi-Fi communication, which is a relatively large capacity communication, can be used for communication of image data, audio data, or the like, and the BLE communication, which is a communication with relatively low power consumption, can be used for communication of control signals or the like. The observation side communication device 140 may further include a communication circuit compatible with cellular phone communication. In addition, the communication between the observation apparatus 100 and the outside of the observation apparatus 100 such as the controller 200 or the thermostatic bath 400 may be performed by wired communication, or may be connected to an electric communication network such as the Internet and performed via the electric communication network such as the Internet. The data transfer between the observation apparatus 100 and the outside of the observation apparatus 100 may be performed via a recording medium outside the observation system 1, such as a USB memory, CD-ROM, a data server on a network, or the like.

As illustrated in FIGS. 1 and 3, the image acquisition unit 150 is provided with an imaging section 151 that images the direction of the sample 300 and acquires the image of the sample 300, and an illumination section 155 that illuminates the sample 300. The imaging section 151 is provided with an imaging optical system and an imaging element, and generates image data based on an image formed on the imaging plane of the imaging element via the imaging optical system. The imaging optical system is preferably a zoom optical system that can change a focal length. The illumination section 155 is provided with an illumination optical system and a light source, and emits illumination light from the light source to the sample 300 via the illumination optical system. The light source is, for example, a light emitting diode (LED). When the observation apparatus 100 is used for observation of cultured cells, it is preferable that the wavelength of the light source is in a red region (for example, a wavelength of 660 nm). As described above, the light source may be appropriately selected according to the observation target. The imaging section 151 is an example of an imaging unit. The image acquisition unit 150 may be expressed as an example of an imaging unit.

The driving mechanism 160 is provided with a support section 165 in which the image acquisition unit 150 is placed, an X feed screw 161 for moving the support section 165 in the X axis direction, and an X actuator 162. In addition, the driving mechanism 160 further includes a Y feed screw 163 for moving the support section 165 in the Y axis direction, and a Y actuator 164. The driving mechanism 160 may be provided with a Z feed screw for moving the support section 165 in the Z axis direction, a Z actuator, and the like.

The sensor section 170 includes a temperature sensor. The temperature sensor is arranged so as to be able to measure the temperature inside the observation side casing 101 of the observation apparatus 100. The temperature sensor may be arranged at a plurality of positions. For example, the temperature sensors may be arranged so that the temperatures of the observation side casing 101 and the transparent plate 102 can be measured. In addition, the temperature sensor may be arranged so that the surface temperature outside the transparent plate 102 and the temperature outside the observation apparatus 100 can be further measured. The sensor section 170 is an example of a temperature sensor.

The clock section 180 generates time information. The time information output from the clock section 180 is used, for example, at the time of recording acquisition data and at the time of determination related to the operation of the observation apparatus 100.

The power source 190 supplies power to each of the elements of the observation apparatus 100. The power source 190 may include, for example, a lithium ion battery or the like, may be a battery connected to an external power source and supplied with power from the outside, or may be a combination of an external power source and a battery. In addition, the power source 190 is provided with a power switch operated at a time when the user wants to turn on the power of the observation apparatus 100. The power source 190 turns on the power of the observation apparatus 100, for example, when the power switch is operated, when the control signal for turning on the power is received from the controller 200 via the observation side communication device 140, and the like.

The outline of the configuration example of the image acquisition unit 150 and the sample 300 of this embodiment is schematically illustrated in FIG. 5, and the image acquisition of this embodiment will be described with reference to FIG. 5. As illustrated in FIG. 5, the illumination section 155 for illuminating the sample 300 is provided on the surface of the support section 165 on the sample 300 side (+Z side), and the imaging section 151 is provided in the vicinity of the illumination section 155. The illumination light emitted from the illumination section 155 illuminates the reflecting plate 360 provided on top of the vessel 310 and is reflected by the reflecting plate 360. The illumination light (reflected light) reflected by the reflecting plate 360 is incident on the imaging section 151 after illuminating the cells 324. The imaging section 151 captures the image formed by the incident light (reflected light). As described above, the image acquisition unit 150 illuminates the sample 300 through the transparent plate 102 and captures to acquire the image of the sample 300. In addition, the relative position between the image acquisition unit 150 and the sample 300 is changed by the driving mechanism 160. As described above, the observation apparatus 100 performs repeated capturing while changing the position of the image acquisition unit 150 in the X direction and the Y direction by the driving mechanism 160, and acquires a plurality of images.

The observation apparatus 100 may perform repeated capturing while changing the imaging position in the thickness (Z axis) direction. The imaging position in the Z-axis direction is changed by changing the focus position of the imaging optical system of the imaging section 151. That is, the imaging optical system is provided with a focus adjustment mechanism for moving a focusing lens an optical axis direction. In addition, the focus adjustment mechanism may be, for example, a lens having a variable focal length such as a liquid lens, or may be provided with a plurality of lenses having different focal lengths. As illustrated in FIG. 5, the range of the focus position is stored in the observation side storage circuit 130, for example, as the focus position range ΔZ. As the focus position range ΔZ, for example, a value corresponding to the size of the sample 300 or the like is preset or set by the user's input. As for the method of acquiring images at a plurality of Z direction positions, the position in the Z axis direction may be fixed and scanned in the X direction and the Y direction, and then the position in the Z axis direction may be changed and scanned in the X direction and the Y direction again. In addition, the capturing may be performed twice or more times while changing the position in the Z axis direction for one position in the X direction and the Y direction, and the imaging may be performed twice or more while scanning in the X direction and the Y direction.

As described above, since the image acquisition unit 150 for generating image data by imaging through the transparent plate 102 and the driving mechanism 160 for moving the image acquisition unit 150 are provided inside the observation side casing 101, it is highly reliable, is easy to handle and clean, and can be structured to prevent contamination and the like.

As described above, for example, as illustrated in FIG. 1, the observation side control circuit 110, the image processing circuit 120, the observation side storage circuit 130, and the observation side communication device 140 are provided inside the observation side casing 101 as an observation side circuit group 104.

### (Controller)

The controller 200 is, for example, a personal computer (PC) or a tablet type information terminal. In FIG. 1, a tablet type information terminal is depicted. As illustrated in FIGS. 1 and 3, the controller 200 is provided with a controller side control circuit 210, a controller side storage circuit 230, a controller side communication device 240, and a controller side input/output device 270.

The controller side control circuit 210 controls operations of each of the elements of the controller 200. The controller side control circuit 210 functions as a system control function 211, a display control function 212, a recording control function 213, and a communication control function 214. Each of the functions 211 to 214 is realized, for example, by causing the controller side control circuit 210 to execute a program stored in the controller side storage circuit 230. The system control function 211 performs various operations related to control for measurement of the sample 300. The display control function 212 controls operations of a display 272. The display control function 212 displays various kinds of information or the like on the display 272. The recording control function 213 controls recording of information in the controller side storage circuit 230. The communication control function 214 controls communication with the observation apparatus 100 through the controller side communication device 240.

The controller side storage circuit 230 stores, for example, programs and various parameters for use in the controller side control circuit 210. In addition, the controller side storage circuit 230 stores data obtained by and received from the observation apparatus 100. Furthermore, the controller side storage circuit 230 temporarily stores various data, for example, received image data (pixel data), image data for recording, image data for display, processing data at the time of operation, and the like.

The controller side communication device 240 is provided with a circuit for the controller 200 to communicate with other devices. The controller side communication device 240 communicates with the observation apparatus 100 through the observation side communication device 140.

The controller side input/output device 270 is provided with the display 272 such as a liquid crystal display and an input device 274 such as a touch panel. In addition to the touch panel, the input device 274 may include a switch, a dial, a keyboard, a mouse, and the like.

### (Thermostatic Bath)

The thermostatic bath 400 is, for example, an incubator used for cell culture or the like. As illustrated in FIG. 2, the thermostatic bath 400 is provided with a thermostatic chamber 402 provided with a shelf 403 on which cultured cells are placed. The shelf 403 includes an upper shelf 403a and a lower shelf 403b. As illustrated in FIGS. 2 and 3, the thermostatic bath 400 of this embodiment is provided with a thermostatic bath side control circuit 410, a temperature control section, a sensor section, a thermostatic bath side communication device 440, a thermostatic bath side storage circuit 450, and a thermostatic bath side input/output device 460. The temperature control section is provided with a heater/fan driving unit 421, a heater 422, a fan 423, and a duct 424. The sensor section is provided with a temperature sensor 431 and a detector 432.

The thermostatic bath side control circuit 410 controls operations of each of the elements of the thermostatic bath 400. The thermostatic bath side control circuit 410 functions as a temperature control function 411, a position acquisition function 412, a temperature acquisition function 413, a display control function 414, and a communication control function 415. Each of the functions 411 to 415 is realized by, for example, causing the thermostatic bath side control circuit 410 to execute a program stored in the thermostatic bath side storage circuit 450. The thermostatic bath side control circuit 410 is an example of second control circuitry. The temperature control function 411 performs temperature control in the thermostatic chamber 402 of the thermostatic bath 400 based on the temperature measured at the reference control position of the thermostatic bath 400 and the set temperature To of the thermostatic bath 400. The set temperature To is the target temperature of the temperature control at the reference control position and can be updated by the indication temperature or the input indication temperature. The position acquisition function 412 acquires position information on the position (control position) at which the observation apparatus 100 is placed within the thermostatic chamber 402 of the thermostatic bath 400. The position information on the control position is information on the height (Z position) of the shelf 403 (the upper shelf 403a and/or the lower shelf 403b) on which the observation apparatus 100 is placed. The acquisition of the control position is performed based on the output of the detector 432, for example. The temperature acquisition function 413 acquires the current temperature at the reference control position based on the output of the temperature sensor 431. The display control function 414 controls the operation of a display 461. The display control function 414 generates display information and displays various kinds of information or the like on the display 461 provided in the thermostatic bath side input/output device 460. The communication control function 415 controls communication with the observation apparatus 100 through the thermostatic bath side communication device 440.

The heater/fan driving unit 421 drives the heater 422 and the fan 423. The heater 422 heats gas that is supplied by the fan 423 in the thermostatic chamber 402. The heater 422 may be, for example, a heater using a heating wire, a ceramic, or the like as a heating element, or may be a Peltier element. The fan 423 supplies the gas in the thermostatic chamber 402 to the heater 422 and blows the gas heated by the heater 422 into the duct 424. The gas heated by the heater 422 passes through the interior of the duct 424 and is supplied to the thermostatic chamber 402 from the lower side (the −Z side) of the thermostatic chamber 402.

The temperature sensor 431 measures a temperature. The temperature sensor 431 is, for example, a thermistor. The temperature sensor 431 may be a thermocouple or a platinum resistance temperature detector. The temperature sensor 431 is arranged so as to be able to measure the temperature at the reference control position in the thermostatic chamber 402 of the thermostatic bath 400. The detector 432 detects whether or not the observation apparatus 100 is placed. The detector 432 is placed in each of the upper shelf 403a and the lower shelf 403b. The detector 432 may be any sensor as long as the sensor can detect that the observation apparatus 100 is placed. The detector 432 is, for example, a strain sensor, and detects whether or not it is pressed by the weight of the observation apparatus 100. The detector 432 is, for example, a brightness sensor, and detects whether or not light is blocked by the observation apparatus 100. The detector 432 is an example of detection circuitry.

The thermostatic bath side communication device 440 is provided with a circuit for the thermostatic bath 400 to communicate with other devices. The thermostatic bath side communication device 440 communicates with the observation apparatus 100 through the observation side communication device 140.

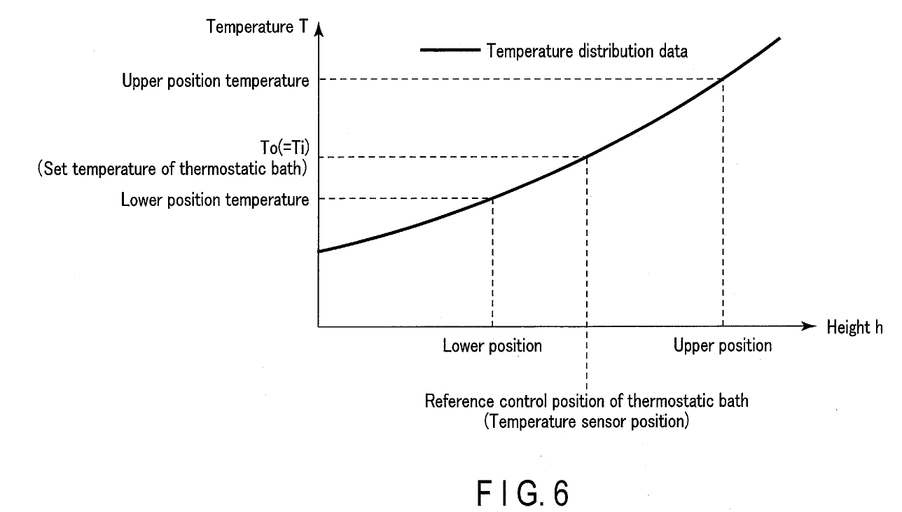

The thermostatic bath side storage circuit 450 stores, for example, programs or various parameters used in the thermostatic bath side control circuit 410. Various data such as received various parameters and processing data at the time of operation are temporarily stored in the thermostatic bath side storage circuit 450. Various parameters stored by the thermostatic bath side storage circuit 450 include temperature distribution data 451, the set temperature To of the thermostatic bath 400, the normal range of the set temperature To, the sample temperature, the appropriate range of the sample temperature, the target temperature (appropriate temperature) of the sample 300, the measured internal temperature of the thermostatic bath, the position (height) in the Z direction in which each detector 432 is placed, and the like. FIG. 6 illustrates a view for explaining the temperature distribution data 451. In the graph illustrated in FIG. 6, a horizontal axis represents a height h, and a vertical axis represents a temperature T. As illustrated in FIG. 6, the temperature distribution data 451 is, for example, data indicating the temperature distribution in the thermostatic chamber 402 of the thermostatic bath 400, and is data indicating a temperature difference occurring between the reference control position (the position of the temperature sensor 431) and the arbitrary position in the thermostatic bath 400. FIG. 6 illustrates an example of a case in which the temperature control of the thermostatic bath 400 is in a steady state and the set temperature To coincides with the measured temperature at the reference control position (the internal temperature Ti of the thermostatic bath). The temperature distribution data 451 is used to calculate the temperature at the reference control position to be set, based on the target upper position temperature and the upper position that is the height of the upper shelf 403a, for example, when the observation apparatus 100 is placed on the upper shelf 403a. The temperature distribution data 451 is set for each type of the thermostatic bath 400, for example. These various parameters may be set in advance, may be set by the user's input in the thermostatic bath 400, or may be set by reception from the observation apparatus 100 or the controller 200. The thermostatic bath side storage circuit 450 is an example of a memory.

The thermostatic bath side input/output device 460 is provided with, for example, a display 461 such as a liquid crystal display and an input device 462 such as a touch panel. Display information for setting various parameters, display information such as the current measured temperature (internal temperature Ti of the thermostatic bath), the current set temperature To of the thermostatic bath 400, and the like are displayed on the display 461. In addition to the touch panel, the input device 462 may include a switch, a dial, a keyboard, a mouse, and the like.

As described above, for example, as illustrated in FIG. 2, the thermostatic bath side control circuit 410, the thermostatic bath side communication device 440, and the thermostatic bath side storage circuit 450 are provided inside a thermostatic bath side casing 401 as a thermostatic bath side circuit group 404.

The observation side control circuit 110, the image processing circuit 120, the controller side control circuit 210, and the thermostatic bath side control circuit 410 include an integrated circuit such as a central processing unit (CPU), an application specific integrated circuit (ASIC), and a field programmable gate array (FPGA). The observation side control circuit 110, the image processing circuit 120, the controller side control circuit 210, and the thermostatic bath side control circuit 410 may each be constituted by one integrated circuit or the like, or my each be constituted by a combination of plurality of integrated circuits or the like. The observation side control circuit 110 and the image processing circuit 120 may be constituted by one integrated circuit or the like. The operation of these integrated circuits is performed according to the program stored in the storage area in the observation side storage circuit 130, the controller side storage circuit 230, the thermostatic bath side storage circuit 450, or the integrated circuit, for example.

The observation side storage circuit 130, the controller side storage circuit 230, and the thermostatic bath side storage circuit 450 are non-volatile memories such as flash memories, but may further have volatile memories such as static random access memory (SRAM) or dynamic random access memory (DRAM). The observation side storage circuit 130, the controller side storage circuit 230, and the thermostatic bath side storage circuit 450 may each be constituted by one memory or the like, or may each be constituted by a combination of a plurality of memories or the like. A database or the like outside the observation system 1 may be used as a part of these memories.

### <Operation of Observation System>

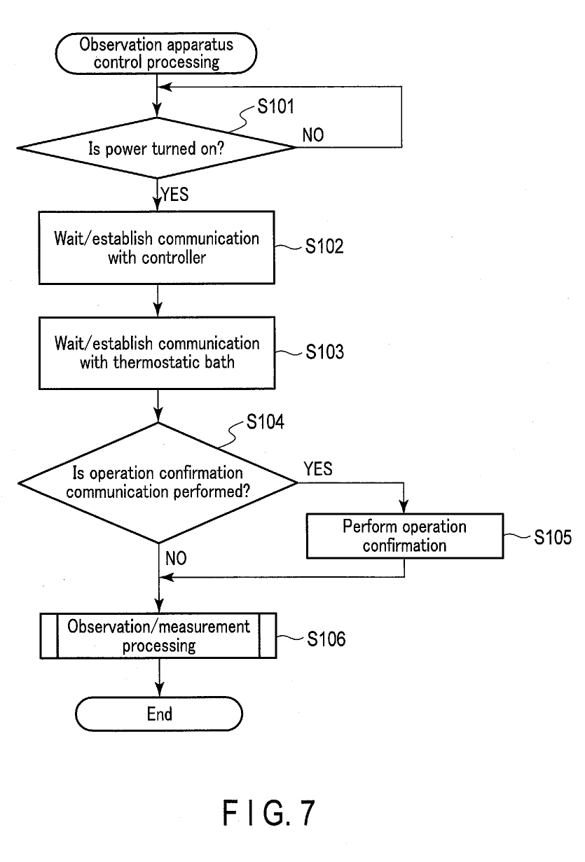

FIG. 7 illustrates an example of observation apparatus control processing executed in the observation apparatus 100 while communicating with the controller 200 and the thermostatic bath 400, and the operation of the observation system 1 will be explained with reference to FIG. 7. The following processing is performed in a state in which, for example, the controller 200 and the thermostatic bath 400 are previously registered as communication partners of the observation apparatus 100. The observation apparatus control processing may be started in a state in which, for example, the BLE communication is in a standby state and the power of the observation apparatus 100 is off, so as to transmit and receive control signals or the like.

In step S101, the observation side control circuit 110 determines whether or not the power of the observation apparatus 100 is turned on. In this determination, it is determined that the power is turned on, for example, when the user operates the power switch of the power source 190, when it is connected to the external power source, when the set time comes, when a control signal for turning on the power is received from the controller 200, or the like. If it is determined that the power is turned on, the processing proceeds to step S102, and if not determined, the processing waits until it is determined that the power is turned on.

In step S102, the observation side control circuit 110 waits until a control signal related to the operation of the observation apparatus 100, various settings, or the like is received from the controller 200. The control circuit 110 establishes communication with the controller 200 after the control signal is received. In step S103, the control circuit 110 establishes communication with the thermostatic bath 400 in the same manner as the processing in step S102, for example.

In step S104, the observation side control circuit 110 determines whether or not the control signal instructing the execution of the operation confirmation has been received from the controller 200. If it is determined that the control signal has been received, the processing proceeds to step S105, and if not determined, the processing proceeds to step S106.

In step S105, the observation side control circuit 110 causes each of the elements of the observation apparatus 100 to execute the operation confirmation according to the received control signal. The operation of each of the elements of the observation apparatus 100 at the time of the operation confirmation may be preset and stored in the observation side storage circuit 130. The result of the operation confirmation such as the usability notification or the malfunction notification may be notified (warned) to the controller 200. For example, before or after the operation confirmation processing, the observation apparatus 100 is placed inside the thermostatic chamber 402 of the thermostatic bath 400.

In step S106, the observation side control circuit 110 executes the observation/measurement processing based on, for example, the control signal corresponding to the operation result of the user received from the controller 200. After the observation/measurement processing is completed, the processing is ended.

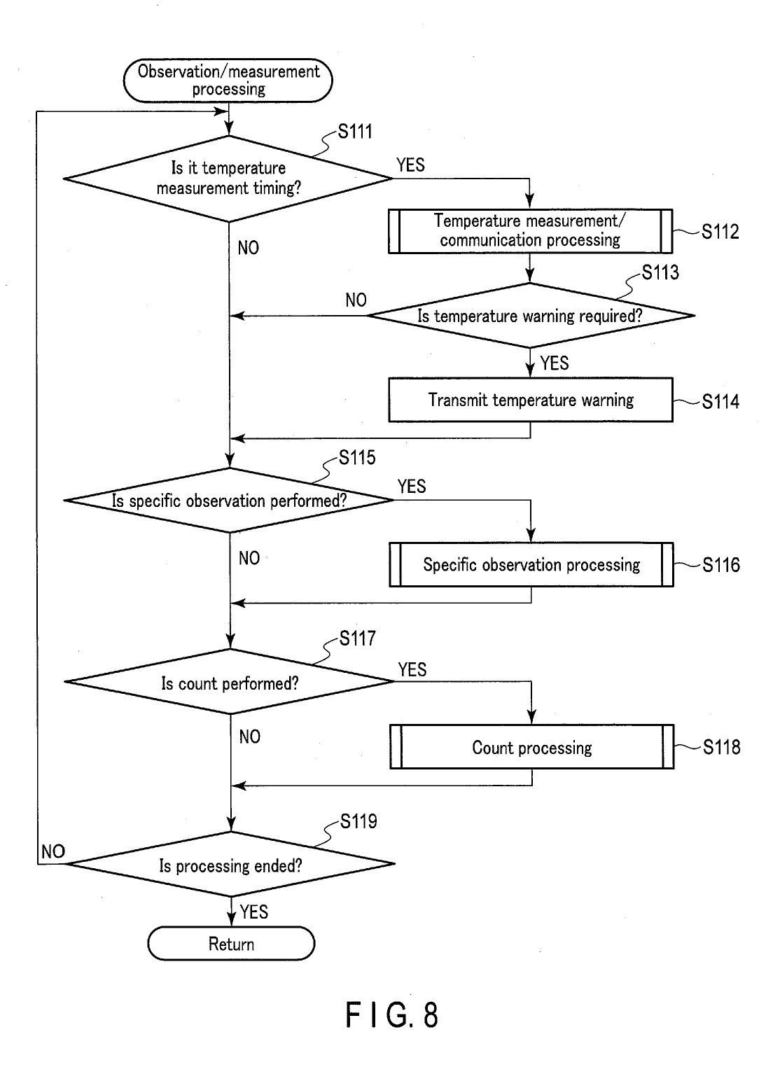

FIG. 8 is a flowchart of an example of the observation/measurement processing in step S106 of the observation apparatus control processing, and the operation of the observation system 1 will be explained with reference to FIG. 8.

In step S111, the observation side control circuit 110 determines whether or not it is a timing to execute the temperature measurement/communication processing. In this determination, it is determined that it is the timing to execute the temperature measurement/communication processing when a predetermined time comes, when a predetermined time interval has elapsed since the execution of the previous temperature measurement/communication processing, or when a control signal instructing to execute the temperature measurement/communication processing is received from the controller 200 or the thermostatic bath 400. The predetermined time, the predetermined time interval, and the like are preset and stored in the observation side storage circuit 130. The timing to execute the temperature measurement/communication processing can be determined according to the performance related to the temperature control of the thermostatic bath 400. If it is determined that it is the timing to execute the temperature measurement/communication processing, the processing proceeds to step S112, and if not determined, the processing proceeds to step S115.

In step S112, the observation side control circuit 110 executes the temperature measurement/communication processing. Details of the temperature measurement/communication processing will be described later.

In step S113, the observation side control circuit 110 determines whether or not the temperature warning is required, based on the result of the temperature measurement/communication processing. If it is determined that the temperature warning is required, the processing proceeds to step S114, and if not determined, the processing proceeds to step S115.

In step S114, the observation side control circuit 110 generates a control signal for executing temperature warning and transmits the control signal to the controller 200 or the thermostatic bath 400. The controller 200 or the thermostatic bath 400 having received the control signal generates and displays display information for performing the temperature warning to the user. The execution of the temperature warning is not limited to the display, but may be performed by a sound such as a warning sound.

In step S115, the observation side control circuit 110 determines whether or not a control signal instructing execution of specific observation processing is received from the controller 200. If it is determined that the control signal instructing the execution of the specific observation processing is received, the processing proceeds to step S116, and if not determined, the processing proceeds to step S117.

In step S116, the observation side control circuit 110 executes processing related to the specific observation. The specific observation is observation and measurement that the user performs by designating a specific position. For example, the user designates the area to be observed by operating the controller 200 while viewing the live view display, or inputs and designates position coordinates indicating the area to the controller 200. The control circuit 110 causes the driving mechanism 160 to move the image acquisition unit 150 according to an operation signal or a coordinate signal based on a user operation received from the controller 200. The control circuit 110 causes the image acquisition unit 150 to capture an image at the position after the movement and causes the observation side communication device 140 to transmit the acquired image to the controller 200. After the specific observation processing is completed, the processing proceeds to step S117.

In step S117, the observation side control circuit 110 determines whether or not a control signal instructing execution of count processing is received from the controller 200. If it is determined that the control signal instructing the execution of the count processing is received, the processing proceeds to step S118. If not determined, the processing proceeds to step S119.

In step S118, the observation side control circuit 110 executes the count processing. In the count processing, the control circuit 110 causes the driving mechanism 160 to move the image acquisition unit 150 according to, for example, a moving pattern or a scan pattern stored in the observation side storage circuit 130. While being moved, the image acquisition unit 150 repeatedly images each predetermined position to acquire an image. The control circuit 110 causes the observation side storage circuit 130 to store the acquired image or the like or causes the image processing circuit 120 or the like to analyze the acquired image. The control circuit 110 counts the number of cells 324, for example, based on the result of analyzing the image, evaluates the states of the cells 324 (for example, whether or not the cell is weak, whether or not the cell is alive, or the like), evaluates the state of the culture medium containing the color of the culture medium, and evaluates whether or not the replacement of the culture medium is required. Based on the results of these evaluations, the control circuit 110 determines whether it is necessary to warn the user, for example, that the replacement of the culture medium is required, the state of the cell is not normal, or the like. The control circuit 110 causes the observation side communication device 140 to transmit the result of the count processing to the controller 200. The result of the count processing includes the acquired image, various composite images obtained by combining the images, the number of cells 324, the states of the cells 324, the state of the culture medium, the control signal instructing the warning to the user, and the like. After the count processing is completed, the processing proceeds to step S119.

In step S119, the observation side control circuit 110 determines whether or not to end the observation/measurement processing according to the result of the user operation, for example. In this determination, for example, when the user instructs the execution of the specific observation or the count processing again, it is determined that the processing is not completed. This determination may be performed based on the operations of each of the elements of the observation apparatus 100 such as the power switch, or may be performed based on the control signal received from the controller 200 or the thermostatic bath 400. If it is not determined that the processing is completed, the processing returns to step S111, and if determined, the observation/measurement processing and the observation apparatus control processing are ended.

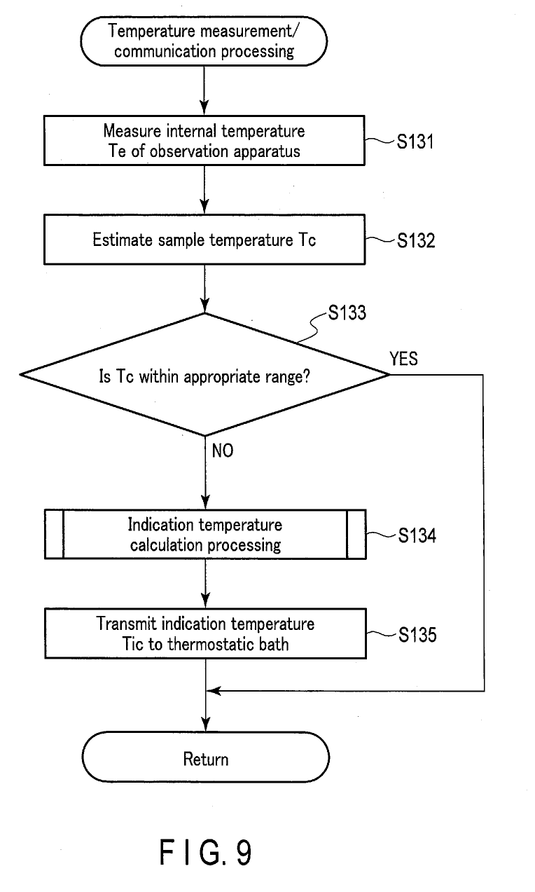

FIG. 9 is a flowchart of an example of the temperature measurement/communication processing in step S112 of the observation/measurement processing, and the operation of the observation system 1 will be explained with reference to FIG. 9.

In step S131, the observation/measurement control function 111 of the observation side control circuit 110 acquires the output of the sensor section 170 and measures the internal temperature Te of the observation apparatus 100.

In step S132, the temperature estimation function 112 of the observation side control circuit 110 estimates the temperature of the sample 300 (the sample temperature Tc) based on the internal temperature Te of the observation apparatus 100 and the temperature estimation formula stored in the observation side storage circuit 130.

In step S133, the observation side control circuit 110 determines whether or not the sample temperature Tc is within an appropriate range. The appropriate range is, for example, the temperature set as the target temperature Tm of the control position ±1° C. The target temperature Tm in the observation system 1 of this embodiment is, for example, 37° C. The control position, the target temperature Tm, the temperature range determined as the appropriate range, and the like are preset and stored in the observation side storage circuit 130 or the like. The value in the appropriate range may be set according to the type of the sample 300 or the type of the thermostatic bath 400. When it is determined that the sample temperature Tc is within the appropriate range, the processing ends the temperature measurement/communication processing to proceed to step S113 of the observation/measurement processing, and if it not determined, the processing proceeds to step S134.

In step S134, the observation side control circuit 110 executes indication temperature calculation processing. The indication temperature calculation processing is processing of calculating an indication temperature Tic indicated to the thermostatic bath 400 as the set temperature To. Details of the indication temperature calculation processing will be described later.

In step S135, the observation side control circuit 110 causes the observation side communication device 140 to transmit the indication temperature Tic calculated in the indication temperature calculation processing to the thermostatic bath 400. For example, communication with low power consumption, such as BLE, may be used for the transmission of the indication temperature Tic. After that, the processing ends the temperature measurement/communication processing and proceeds to step S113 of the observation/measurement processing.

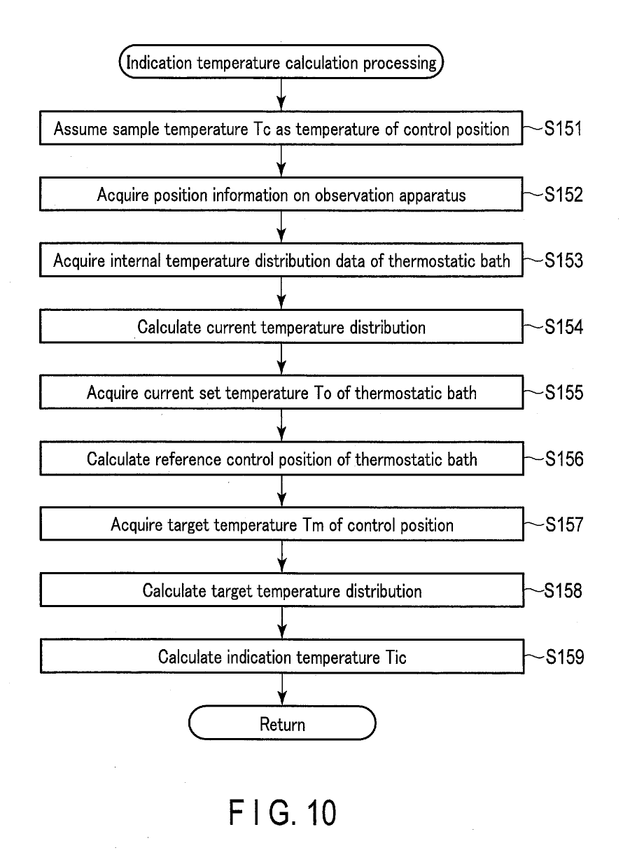

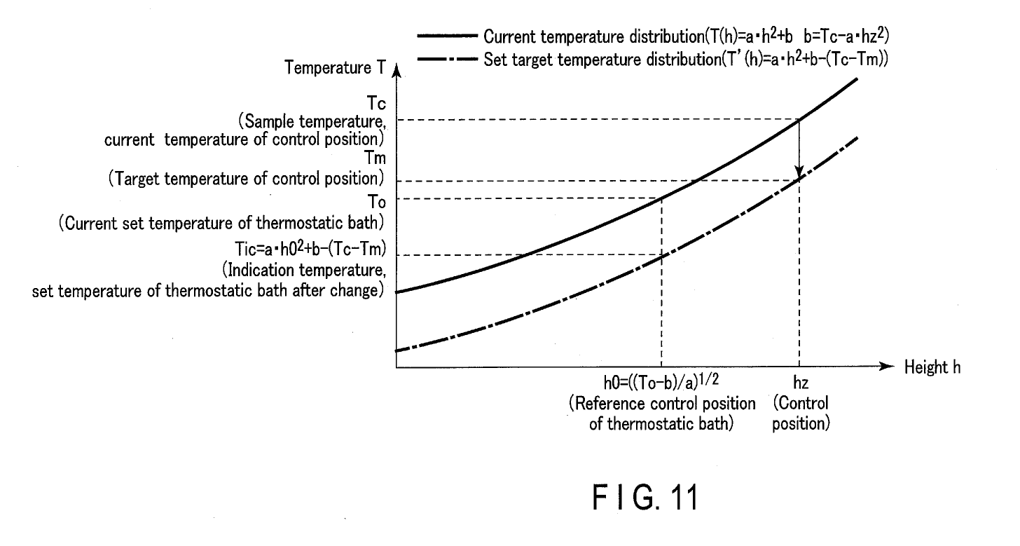

FIG. 10 is a flowchart of an example of the indication temperature calculation processing in step S134 of the temperature measurement/communication processing, and FIG. 11 is a view for explaining the indication temperature calculation processing. The operation of the observation system 1 will be explained with reference to FIGS. 10 and 11.

In step S151, the observation side control circuit 110 acquires the estimated sample temperature Tc as the current temperature at the control position at which the observation apparatus 100 is placed. When the temperature control of the thermostatic bath 400 is in a steady state, the temporal change in the internal temperature of the thermostatic bath 400 is small. Therefore, the temperature at the control position and the sample temperature Tc can be regarded as equal to each other. Therefore, the observation system 1 of this embodiment treats the sample temperature Tc as the current temperature at the control position, and performs temperature control inside the thermostatic bath 400.

In step S152, the observation side control circuit 110 acquires position information on the position (control position) at which the observation apparatus 100 is placed. The position information on the control position is acquired based on, for example, the output of the detector 432 of the thermostatic bath 400. For example, when the observation apparatus 100 is placed on the upper shelf 403a among the plurality of shelves 403 (the upper shelf 403a and the lower shelf 403b) provided in the thermostatic bath 400, the observation side control circuit 110 acquires the height hz of the upper shelf 403a as the position information on the control position.

In step S153, the observation side control circuit 110 acquires temperature distribution data 451 stored in the thermostatic bath side storage circuit 450 of the thermostatic bath 400.

In step S154, the observation side control circuit 110 calculates a current temperature distribution T(h) based on the temperature distribution data 451, the sample temperature Tc, and the position information (height hz) of the control position. Here, h is the height (Z direction position) in the thermostatic chamber 402. An example of the current temperature distribution T(h) calculated in this step is indicated by a solid line in the graph of FIG. 11. For example, a case in which the temperature distribution indicated by the temperature distribution data is expressed by Equation (1) below will be described:

T(h)=a×h²+b Equation (1).

At this time, the sample temperature Tc and the position information (height hz) of the control position are used to calculate the constant b as follows:

b=Tc−a×(hz)² Equation (2).

At this time, for example, as the temperature distribution data 451, the fact that the temperature distribution T(h) is the quadratic function of the height h, the proportionality constant of the term of h² is a, the proportionality constant of the term of h is 0, and the constant term is present is stored in the thermostatic bath side storage circuit 450. For example, the value of the proportionality constant a can be set for each type of the thermostatic bath 400, each set temperature To of the thermostatic bath 400, and each position (X position or Y position) in the thermostatic chamber 402. In this embodiment, a case in which the temperature distribution T(h) is the quadratic function of the height h is explained as an example, but the present invention is not limited thereto. The temperature distribution T(h) may be a linear function of the height h, or may be a cubic or higher-order function.

In step S155, the observation side control circuit 110 acquires the current set temperature To of the thermostatic bath 400. The set temperature To is acquired from, for example, the thermostatic bath 400 through communication.

In step S156, the observation side control circuit 110 calculates the reference control position h0 of the thermostatic bath 400 based on the current set temperature To of the thermostatic bath 400 and the current temperature distribution T(h). This calculation is based on the assumption that the current temperature control in the thermostatic bath 400 is in the steady state and the current temperature at the reference control position h0 is the set temperature To. At this time, from Equations (1) and (2), the reference control position h0 is calculated as follows:

h0={(To−Tc)/a+(hz)²}^{1/2} Equation (3).

In step S157, the observation side control circuit 110 acquires the target temperature Tm at the control position (height hz). The target temperature Tm is previously set and is obtained from, for example, the thermostatic bath side storage circuit 450 of the thermostatic bath 400.

In step S158, the observation side control circuit 110 calculates the target temperature distribution. T′(h) based on the target temperature Tm at the control position (height hz) and the current temperature distribution T(h). An example of the target temperature distribution T′(h) calculated in this step is indicated by a one-dot chain line in the graph of FIG. 11. For example, the target temperature distribution T′(h) is expressed using the constant b as follows:

T′(h)=a×h²+b−(Tc−Tm) Equation (4).

In step S159, the observation side control circuit 110 calculates the indication temperature Tic, which is the temperature indicated as the set temperature To in the thermostatic bath 400, based on the target temperature distribution T′(h) and the reference control position h0. The indication temperature Tic can also be expressed as the set temperature To of the thermostatic bath 400 after the change by the indication temperature calculation processing of this embodiment. At this time, from Equations (2), (3), and (4), the indication temperature Tic is calculated as follows:

Tic=a×{(h0)²−(hz)²}+Tm=To−(Tc−Tm) Equation (5).

After that, the processing ends the indication temperature calculation processing and proceeds to step S135 of the temperature measurement/communication processing.

A case in which various parameters used for the calculation of the indication temperature Tic are acquired as needed at each step is explained as an example, but the present invention is not limited thereto. Various parameters used for the calculation of the indication temperature Tic may be previously obtained and stored in the observation side storage circuit 130 or the like prior to the indication temperature calculation processing.

A case in which the position information on the control position is acquired based on the output of the detector 432 of the thermostatic bath 400 has been explained as an example, but the present invention is not limited thereto. The position information on the control position may be obtained based on the user's input to the observation apparatus 100, the controller 200, or the thermostatic bath 400. As the position information on the control position, not only the position in the Z direction but also the positions in the X direction and the Y direction may be further acquired. In this case, the temperature distributions may be prepared according to the X positions and the Y positions. The temperature distribution for each type of the thermostatic bath 400 may be registered in the observation apparatus 100 or the controller 200.

A case in which the user previously inputs the target temperature Tm to the thermostatic bath 400 and the input target temperature Tm is stored in the thermostatic bath side storage circuit 450 of the thermostatic bath 400 has been explained as an example, but the present invention is not limited thereto. The target temperature Tm may be acquired based on the user's input to the observation apparatus 100 or the controller 200. The target temperature Tm may be determined based on the input of the type of the sample 300 from the plurality of target temperatures Tm that are previously registered according to the type of the sample 300 (the cells 324 or the vessel 310).

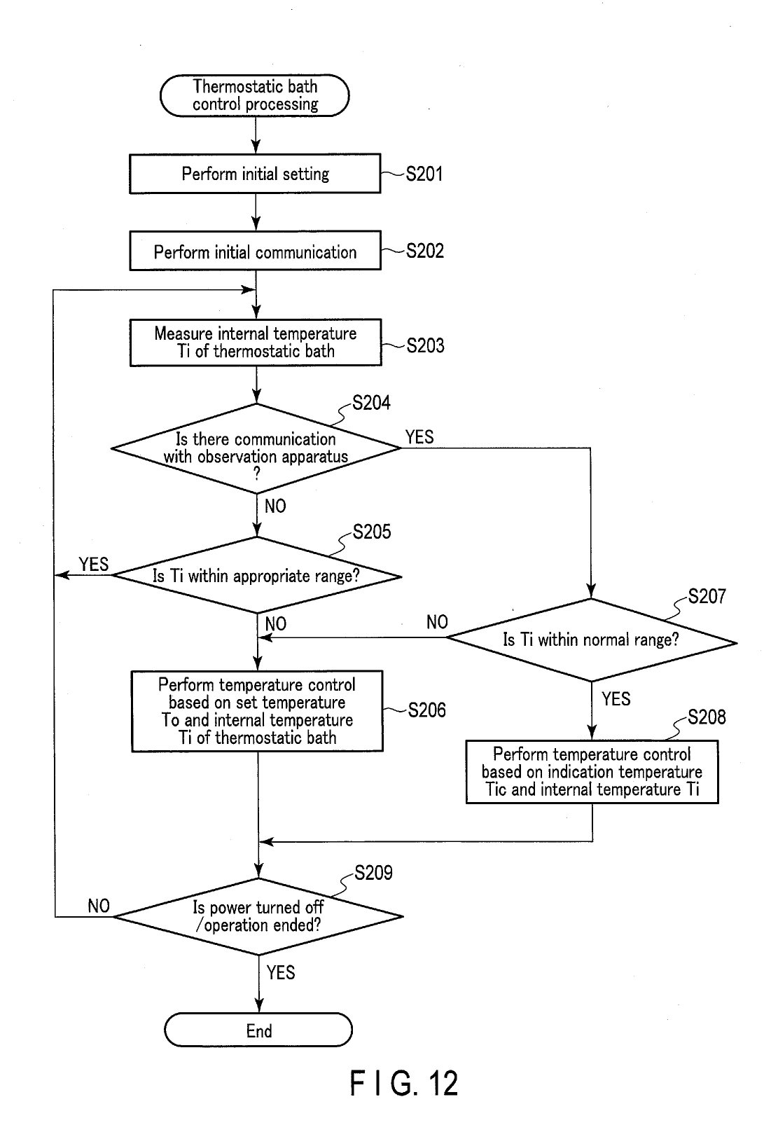

FIG. 12 is a flowchart of an example of thermostatic bath control processing executed in the thermostatic bath 400, and the operation of the observation system 1 will be described with reference to FIG. 12.

In step S201, the thermostatic bath side control circuit 410 performs initial setting. In the initial setting, the control circuit 410 initializes each circuit or the like provided in the thermostatic bath 400. The control circuit 410 sets, as the initial set temperature To, the set temperature To of the thermostatic bath 400 stored in the thermostatic bath side storage circuit 450 by the user's input.

In step S202, the thermostatic bath side control circuit 410 performs initial communication. In the initial communication, the control circuit 410 establishes communication with the observation apparatus 100. At this time, the temperature distribution data 451 may be transmitted to the observation apparatus 100. The processing of this step corresponds to step S103 of the observation apparatus control processing.

In step S203, the thermostatic bath side control circuit 410 measures the internal temperature Ti of the thermostatic bath. The internal temperature Ti is the temperature at the reference control position of the thermostatic bath 400 and is acquired based on the output of the temperature sensor 431. When a plurality of temperature sensors 431 is provided, the temperature measured in the vicinity of the center of the thermostatic chamber 402 among a plurality of measured temperatures may be used as the internal temperature Ti, and the temperature measured at the position closest to the control position may be used as the internal temperature Ti. An average value of the plurality of measured temperatures may be used as the internal temperature Ti.

In step S204, the thermostatic bath side control circuit 410 determines whether or not there is the communication with the observation apparatus 100. If it is determined that there is the communication, the processing proceeds to step S207, and if not determined, the processing proceeds to step S205.

In step S205, the thermostatic bath side control circuit 410 determines whether or not the measured internal temperature Ti of the thermostatic bath 400 is within an appropriate range. The value of the appropriate range is stored in, for example, the thermostatic bath side storage circuit 450. The value of the appropriate range may be acquired from the observation apparatus 100. If it is determined that the value is within the appropriate range, the processing returns to step S203, and if not determined, the processing proceeds to step S206.

In step S206, the thermostatic bath side control circuit 410 performs temperature control based on the set temperature To and the internal temperature Ti of the thermostatic bath 400. At this time, the control circuit 410 performs the temperature control so that the internal temperature Ti becomes equal to the set temperature To, or the internal temperature Ti is within the appropriate range with respect to the set temperature To. After that, the processing proceeds to step S209.

In step S207, the thermostatic bath side control circuit 410 determines whether or not the measured internal temperature Ti of the thermostatic bath is within a normal range. The value of the normal range is stored in, for example, the thermostatic bath side storage circuit 450. The value of the normal range may be acquired from the observation apparatus 100. In this step, the control circuit 410 may transmit the temperature distribution data 451 to the observation apparatus 100. If it is determined that the value is within the normal range, the processing proceeds to step S208, and if not determined, the processing proceeds to step S206. If it is determined that the internal temperature Ti measured in this step is not within the normal range, the temperature control based on the indication temperature Tic is stopped, and the temperature control is performed based on the set temperature To and the internal temperature Ti.

In step S208, the thermostatic bath side control circuit 410 performs the temperature control based on the indication temperature Tic received from the observation apparatus 100 and the internal temperature Ti of the thermostatic bath 400. After that, the processing proceeds to step S209. At this time, the control circuit 410 performs the temperature control so that the internal temperature Ti becomes equal to the indication temperature Tic.

In step S209, the thermostatic bath side control circuit 410 determines whether or not there is an operation to turn off the power, or, for example, an instruction to end the operation is issued from the controller 200. When it is determined that there is the operation to turn off the power or the instruction to end the operation, the operation related to the temperature control or the like of the thermostatic bath 400 is ended to complete the thermostatic bath control processing, and if not determined, the processing returns to step S203.

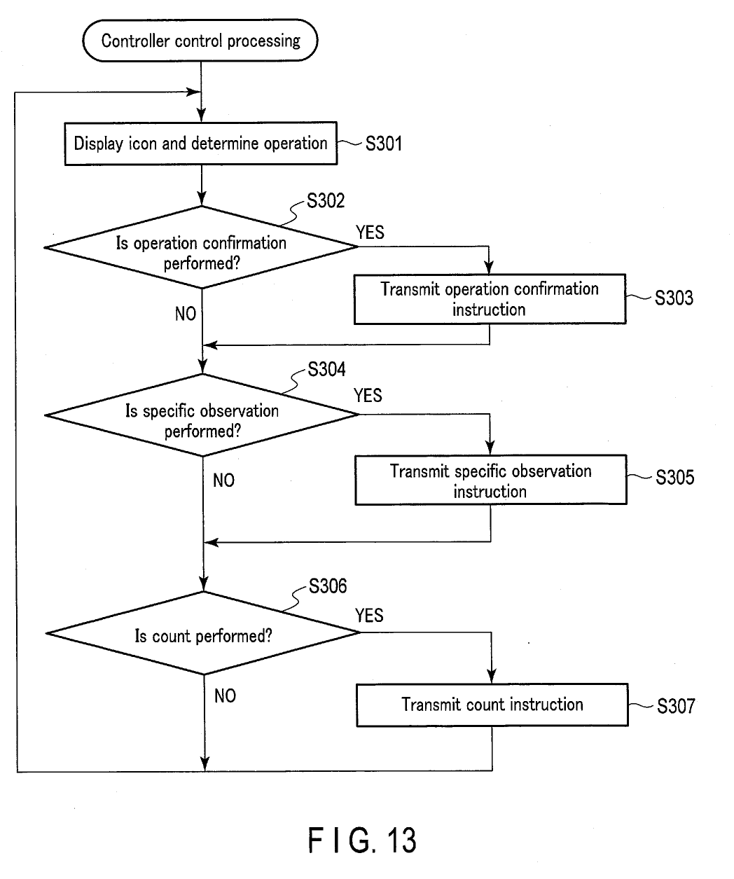

FIG. 13 is a flowchart of an example of the controller control processing executed by the controller 200, and the operation of the observation system 1 will be explained with reference to FIG. 13. The controller control processing illustrated in FIG. 13 is started, for example, after the power of the observation apparatus 100 is turned on.

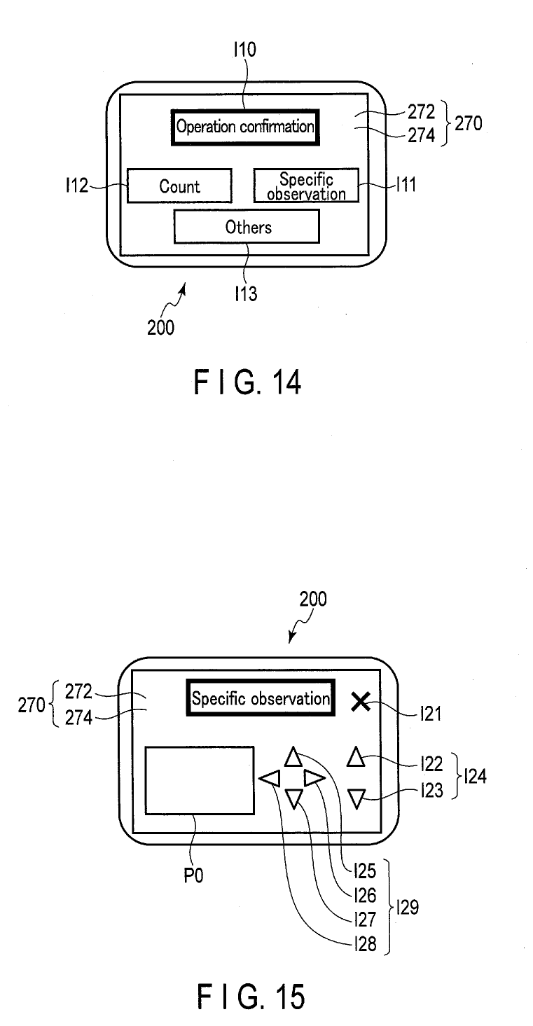

In step S301, the controller side control circuit 210 causes the display 272 to display an icon group (basic icon) for the user to operate the observation apparatus 100. An example of the display at this time is illustrated in FIG. 14. In the display example illustrated in FIG. 14, an operation confirmation icon I10, a specific observation icon I11, a count icon I12, and other icons I13 are displayed. The operation confirmation icon I10 is an icon for instructing the execution of the operation confirmation. The specific observation icon I11 is an icon for instructing the execution of the specific observation. The count icon I12 is an icon for instructing the execution of the count processing of performing cell counting or the like. The other icon I13 is an icon for instructing the execution of other functions or the execution of various settings. As in the display example of the operation confirmation icon I10 illustrated in FIG. 14, the selected icon may be emphasized and displayed. The controller side control circuit 210 determines (operation determination) whether or not the user has selected the icon based on the operation signal output by the input device 274 according to the user operation result. The processing waits until it is determined that the user has selected the icon. If it is determined that the user has selected the icon, the processing proceeds to step S302.

In step S302, the controller side control circuit 210 determines whether or not the operation confirmation icon I10 has been selected. If it is determined that the operation confirmation icon I10 has been selected, the processing proceeds to step S303, and if not, the processing proceeds to step S304.

In step S303, the controller side control circuit 210 establishes communication with the observation apparatus 100. The established communication may be, for example, low power consumption communication for transmitting an instruction to turn on the power of the observation apparatus 100. The control circuit 210 transmits a control signal related to the execution of the operation confirmation to the observation apparatus 100. The operation of the observation apparatus 100 at the time of the operation confirmation may be determined according to the user's operation, or may be preset and stored in the observation side storage circuit 130 or the controller side storage circuit 230. For example, in step S201, the user can select the other icon I13 and set the operation contents or the like. In this step, various parameters used in the observation apparatus 100 are set or transmitted to the observation apparatus 100.

In step S304, the controller side control circuit 210 determines whether or not the specific observation icon I11 has been selected. If it is determined that the specific observation icon I11 has been selected, the processing proceeds to step S305, and if not, the processing proceeds to step S306.

In step S305, when the communication with the observation apparatus 100 is not established, the controller side control circuit 210 establishes the communication. The control circuit 210 generates display information for the specific observation and displays the display information on the display 272. FIG. 15 is a schematic view illustrating an example of the display at this time. In the display example illustrated in FIG. 15, a movement icon I29 for moving the image acquisition unit 150, a focus adjustment icon I24 for performing focus adjustment, a display position P0 for displaying a received image, and an end icon I21 for instructing the end of the specific observation are displayed. The movement icon I29 includes a +Y movement icon I25 for moving in the +Y direction, a +X movement icon I26 for moving in the +X direction, a −Y movement icon I27 for moving in the −Y direction, and an −X movement icon I28 for moving in the −X direction. The focus adjustment icon I24 includes an icon I22 for driving the lens to the infinity side and an icon I23 for driving the lens toward the closest side. The control circuit 210 acquires the image captured by the observation apparatus 100 and displays the captured image on the display 272. The acquired image is displayed at the display position P0 in the display example of FIG. 15, for example. For example, while viewing the live view display displayed at the display position P0, the user operates the movement icon I29 to move the image acquisition unit 150. In addition, the user can adjust the focus state at the observation position by moving the focusing lens or the like in the Z direction by the focus adjustment icon I24 and observe the appearance of the cell or the like at the observation position under a desired focus state. The display at the time of the specific observation may further include an imaging icon instructing the storage of the image, position information indicating the current imaging position of the sample 300, and the like.

In step S306, when the communication with the observation apparatus 100 is not established, the controller side control circuit 210 establishes the communication. The control circuit 210 determines whether or not the count icon I12 has been selected. If it is determined that the count icon I12 has been selected, the processing proceeds to step S307, and if not determined, the processing returns to step S301.

In step S307, the controller side control circuit 210 transmit a control signal instructing the execution of the count processing to the observation apparatus 100. The control circuit 210 displays, on the display 272, the image captured by the image acquisition unit 150 during the count processing. The control circuit 210 displays, on the display 272, the image acquired from the observation apparatus 100, the result of count processing, or the like. In addition, based on the information obtained from the observation apparatus 100, the warning is displayed as necessary, such as whether or not the replacement of the culture medium is required. The determination as to whether or not the replacement of the culture medium is required, or the like may be performed by the controller 200. After that, the processing returns to step S301.

As described above, the observation system 1 of this embodiment calculates the indication temperature Tic indicated as the set temperature To of the thermostatic bath 400 based on the current sample temperature Tc estimated by the observation apparatus 100. Therefore, in the observation system 1 of this embodiment, even when the internal temperature of the thermostatic chamber 402 is not uniform, the appropriate temperature control in the thermostatic chamber 402 can be realized. In addition, in the observation system 1 of this embodiment, even when the thermostatic bath 400 cannot directly measure the temperature of the control position, the appropriate temperature control in the thermostatic chamber 402 can be realized.

### Second Embodiment

A second embodiment of the present invention will be explained. A difference from the first embodiment will be explained. The same parts are denoted by the same reference numerals, and a description thereof will be omitted. In the first embodiment, the observation system 1 in which the observation apparatus 100 communicates with the thermostatic bath 400 and the thermostatic bath 400 can perform the appropriate temperature control based on the indication temperature Tic calculated by the observation apparatus 100 has been explained as an example. On the other hand, the same temperature control can be realized even when the observation apparatus 100 and the thermostatic bath 400 do not communicate with each other. Therefore, in this embodiment, a case in which the user indicates the indication temperature Tic to the thermostatic bath 400 will be explained as an example.

### <Configuration of Observation System>

In the observation system 1 of this embodiment, the observation apparatus 100 communicates with the controller 200. On the other hand, unlike the first embodiment, in the observation system 1 of this embodiment, the observation apparatus 100 and the thermostatic bath 400 do not communicate with each other. The observation side communication device 140 of this embodiment is an example of communication circuitry.

In the observation side storage circuit 130 of the observation apparatus 100 of this embodiment, various parameters required for the calculation of the indication temperature Tic are preset or input by the user and stored. The observation side storage circuit 130 of this embodiment is an example of a memory. Since the observation apparatus 100 communicates with the controller 200, the user's input may be performed by either the observation apparatus 100 or the controller 200. At this time, the observation side communication device 140 of the observation apparatus 100 can be expressed as an example of an input interface. The observation apparatus 100 may further include an input device for inputting various parameters. At this time, the input device of the observation apparatus 100 can be expressed as an example of an input interface. The input device of the observation apparatus 100 is similar to, for example, the input device 274 of the controller 200 and the input device 462 of the thermostatic bath 400. For example, temperature distribution data 451 or the like may be acquired through an electric communication network such as the Internet. The observation apparatus 100 of this embodiment further includes a display for displaying the calculated indication temperature Tic or the like. The display of the observation apparatus 100 is the same as, for example, the display 461 of the thermostatic bath 400 according to the first embodiment.

The controller 200 of this embodiment is the same as the controller 200 according to the first embodiment.

The input device 462 provided in the thermostatic bath 400 of this embodiment further acquires the input indication temperature Tim used as the set temperature To in the thermostatic bath 400. The thermostatic bath 400 of this embodiment is configured so that the display of the observation apparatus 100 placed in the thermostatic chamber 402 is visible from the outside. As the configuration for this, for example, a transparent window is provided in a part of the thermostatic bath side casing 401 of the thermostatic bath 400 of this embodiment.

### <Operation of Observation System>

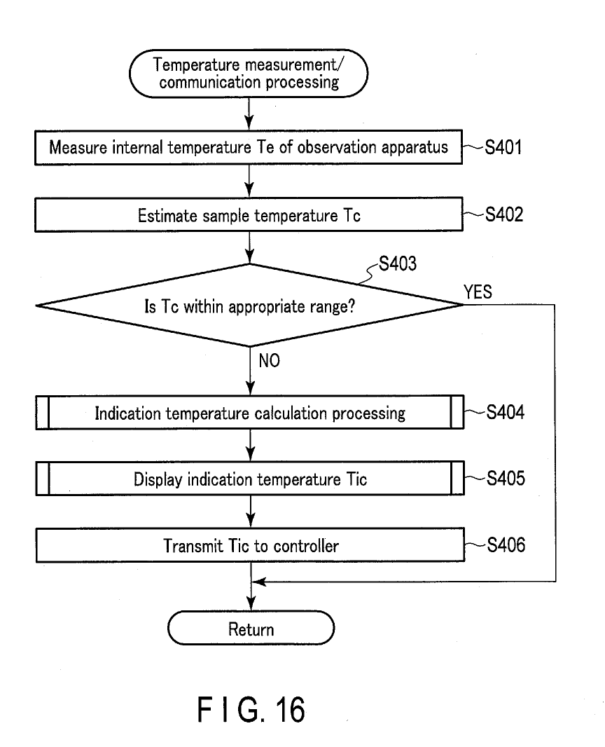

FIG. 16 is a flowchart of an example of temperature measurement/communication processing of this embodiment executed by the observation apparatus 100, and the operation of the observation system 1 will be explained with reference to FIG. 16. The following description is given through the comparison with the temperature measurement/communication processing according to the first embodiment described above with reference to FIG. 9.

In steps S401 to S404, the observation side control circuit 110 measures the internal temperature Te of the observation apparatus 100 and estimates the sample temperature Tc based on the measured internal temperature Te in the same manner as in steps S131 to S134. The control circuit 110 determines whether or not the estimated sample temperature Tc is within an appropriate range. If it is not determined that the estimated sample temperature Tc is within the appropriate range, the control circuit 110 performs the indication temperature calculation processing and calculates the indication temperature Tic. Various parameters required for the calculation of the indication temperature Tic of the control position or the like may be acquired by the input device provided in the observation apparatus 100, or may be acquired by the input device 274 of the controller 200. In step S405, the control circuit 110 displays the indication temperature Tic on the display provided in the observation apparatus 100. In step S406, the control circuit 110 transmits the calculated indication temperature Tic to the controller 200. At this time, the received indication temperature Tic may be displayed in the controller 200. Due to the processing of step S405 or step S406, the user can confirm the indication temperature Tic calculated by the observation apparatus 100. When the calculation of the indication temperature Tic is performed, the sample temperature Tc is outside the appropriate range. Therefore, the observation apparatus 100 or the controller 200 may give a warning to the user prior to or in conjunction with the display of the indication temperature Tic. When it is determined that the calculated sample temperature Tc is within the appropriate range and after the processing of step S406 is completed, the temperature measurement/communication processing is ended.

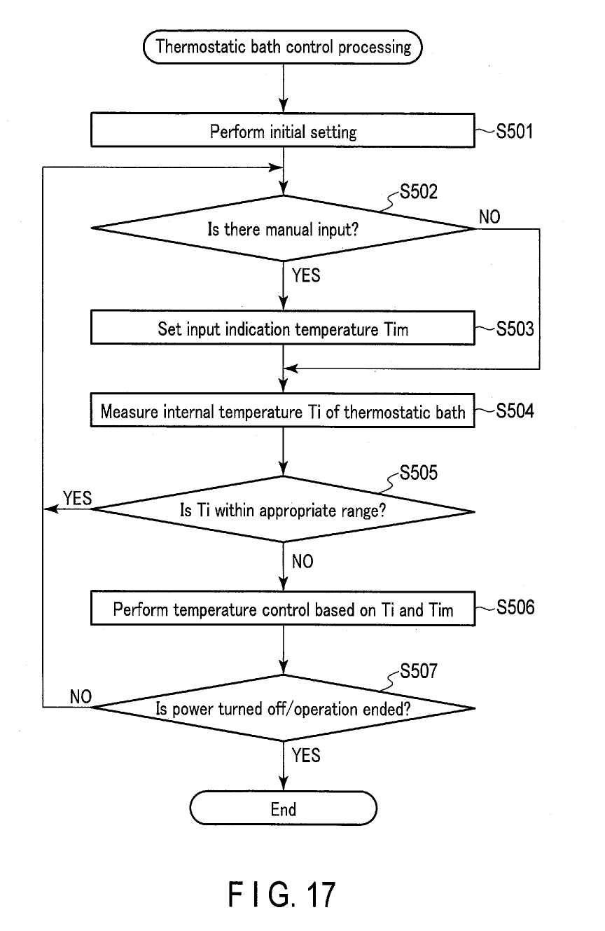

FIG. 17 is a flowchart of an example of thermostatic bath control processing of this embodiment executed by the thermostatic bath 400, and the operation of the observation system 1 will be explained with reference to FIG. 17. The following description is given through the comparison with the thermostatic bath control processing according to the first embodiment described above with reference to FIG. 12.

In step S501, as in step S201, the thermostatic bath side control circuit 410 performs initial setting. In this initial setting, the set temperature To of the thermostatic bath 400 is set based on the user's input or the like, or the set temperature To is set as the initial value of the input indication temperature Tim which is the indication temperature Tic input by the user. In step S502, the control circuit 410 determines whether or not the indication temperature Tic has been manually input based on the output of the input device 462 according to the user operation. If it is determined that the indication temperature Tic has been manually input, the processing proceeds to step S503, and if not determined, the processing proceeds to step S504. In step S503, the control circuit 410 acquires the input indication temperature Tic as the input indication temperature Tim and sets the input indication temperature Tim as the set temperature To of the reference control position. In step S504, the control circuit 410 measures the internal temperature Ti of the thermostatic bath 400 in the same manner as in step S203. In step S505, the control circuit 410 determines whether or not the measured internal temperature Ti is within the appropriate range in the same manner as in step S205. If it is determined that the measured internal temperature Ti is within the appropriate range, the processing returns to step S502, and if not determined, the processing proceeds to step S506. In step S506, the control circuit 410 executes temperature control based on the input indication temperature Tim and the internal temperature Ti in the same manner as in step S208, for example. In step S507, the control circuit 410 determines whether or not to end the thermostatic bath control processing in the same manner as in step S209. If it is determined that the thermostatic bath control processing is ended, the processing is ended, and if not determined, the processing returns to step S502.

As described above, in the observation system 1 of this embodiment, even if the communication is not performed between the observation apparatus 100 and the thermostatic bath 400, the appropriate temperature control in the thermostatic chamber 402 can be realized. Therefore, by using the observation apparatus 100 of this embodiment, the user can observe the cell culture and the sample under the appropriate temperature control, regardless of the type or performance of the thermostatic bath 400.

### Third Embodiment

A third embodiment of the present invention will be explained. A difference from the first embodiment will be mainly explained. The same parts are denoted by the same reference numerals, and a description thereof will be omitted. In the first embodiment, the observation system 1 in which the observation apparatus 100 communicates with the thermostatic bath 400 and the thermostatic bath 400 can perform the appropriate temperature control based on the indication temperature Tic calculated by the observation apparatus 100 has been explained as an example. On the other hand, there may be a case in which the controller 200 communicates with the thermostatic bath 400 and the controller 200 calculates the indication temperature Tic.

### <Configuration of Observation System>

In the observation system 1 of this embodiment, the observation apparatus 100 performs first communication with the controller 200. On the other hand, unlike the first embodiment, in the observation system 1 of this embodiment, the controller 200 and the thermostatic bath 400 perform second communication, and the observation apparatus 100 and the thermostatic bath 400 do not communicate with each other.

The control circuit 110 of the observation apparatus 100 of this embodiment does not function as the indication temperature calculation function 113. On the other hand, the control circuit 210 of the controller 200 of this embodiment functions as the indication temperature calculation function 113 according to the first embodiment. The thermostatic bath 400 of this embodiment is the same as the thermostatic bath 400 according to the first embodiment. The thermostatic bath 400 sets the set temperature To based on the indication temperature Tic received from the controller 200 and performs the temperature control.

### <Operation of Observation System>

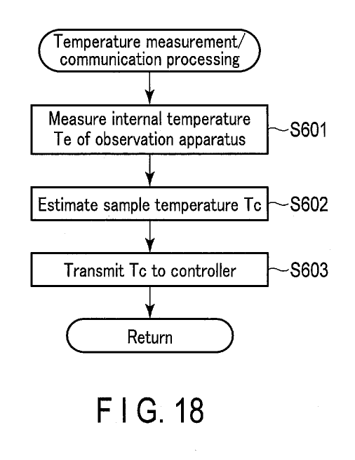

FIG. 18 is a flowchart of an example of temperature measurement/communication processing of this embodiment executed by the observation apparatus 100, and the operation of the observation system 1 will be explained with reference to FIG. 18. The following description is given through the comparison with the temperature measurement/communication processing according to the first embodiment described above with reference to FIG. 9.

In steps S601 and S602, the observation side control circuit 110 measures the internal temperature Te of the observation apparatus 100 and estimates the sample temperature Tc based on the measured internal temperature Te in the same manner as in steps S131 and S132. In step S603, the control circuit 110 transmits the estimated sample temperature Tc to the controller 200. In this step, in the same manner as in step S133, it may be determined whether or not the estimated sample temperature Tc is within the appropriate range. If it is determined that the sample temperature Tc is within the appropriate range, the set temperature To of the thermostatic bath 400 need not be updated, and thus the sample temperature Tc may not be transmitted to the controller 200. After the processing of this step is completed, the temperature measurement/communication processing is ended.

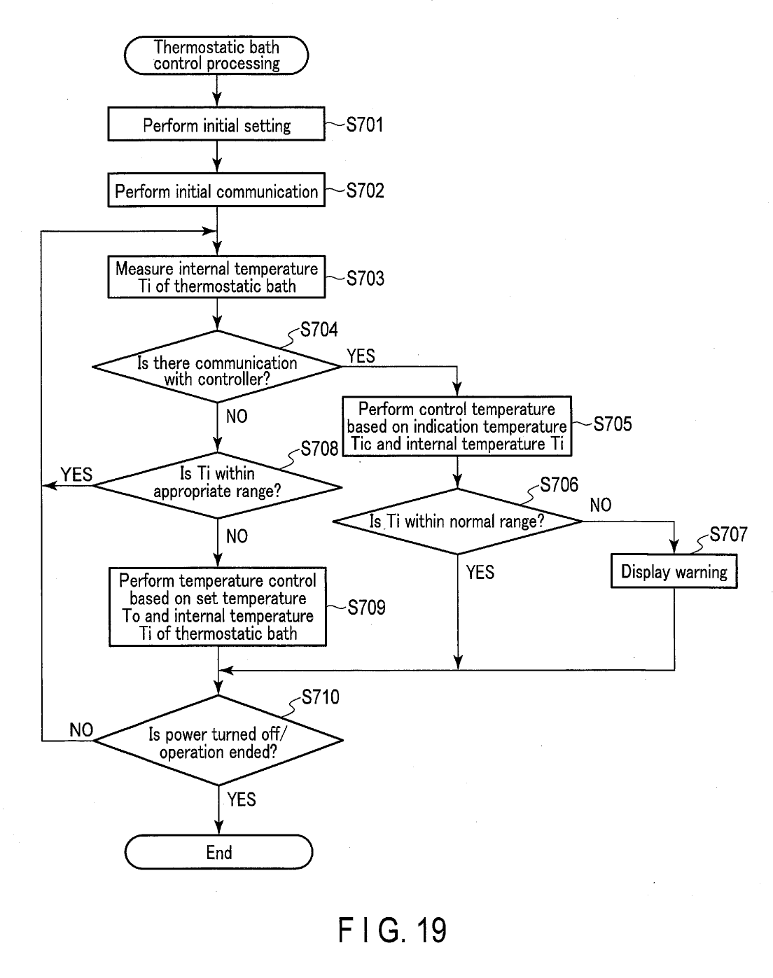

FIG. 19 is a flowchart of an example of thermostatic bath control processing of this embodiment executed by the thermostatic bath 400, and the operation of the observation system 1 will be explained with reference to FIG. 19. The following description is given through the comparison with the thermostatic bath control processing according to the first embodiment described above with reference to FIG. 12.

In steps S701 to S703, the thermostatic bath side control circuit 410 measures the internal temperature Ti of the thermostatic bath 400 after the initial setting and initial communication are performed, in the same manner as in steps S201 to S203. The initial communication in step S702 is performed between the observation apparatus 100 and the controller 200. At this time, the temperature distribution data 451 can be transmitted to the controller 200.

In step S704, the thermostatic bath side control circuit 410 determines whether or not there is the communication from the controller 200, for example, unlike step S204. If it is determined that there is the communication, the processing proceeds to step S705, and if not determined, the processing proceeds to step S708.

In step S705, the thermostatic bath side control circuit 410 performs temperature control based on the indication temperature Tic received from the controller 200 and the internal temperature Ti of the thermostatic bath 400 in the same manner as in step S208, for example.

In step S706, the thermostatic bath side control circuit 410 determines whether or not the measured internal temperature Ti of the thermostatic bath 400 is within the normal range in the same manner as in step S207. If it is determined that the measured internal temperature Ti is within the normal range, the processing proceeds to step S710, and if not determined, the processing proceeds to step S707.

In step S707, the thermostatic bath side control circuit 410 warns the user that the internal temperature Ti of the thermostatic bath 400 is not within the normal range. For example, the warning is performed on the display 461 of the thermostatic bath 400, but a control signal related to the warning may be transmitted to the observation apparatus 100 or the controller 200, and the warning may be performed on the display of the observation apparatus 100 or the display 272 of the controller 200. After that, the processing proceeds to step S710.

In step S708, the thermostatic bath side control circuit 410 determines whether or not the measured internal temperature Ti of the thermostatic bath 400 is within the appropriate range in the same manner as in step S205. If it is determined that the measured internal temperature Ti is within the appropriate range, the processing returns to step S703, and if not determined, the processing proceeds to step S709.

In step S709, the thermostatic bath side control circuit 410 performs temperature control based on the set temperature To and the internal temperature Ti of the thermostatic bath 400 in the same manner as in step S206. After that, the processing proceeds to step S710.

In step S710, the thermostatic bath side control circuit 410 determines whether or not there is an operation to turn off the power, or, for example, an instruction to end the operation is issued from the controller 200 in the same manner as in step S209. When it is determined that there is the operation to turn off the power, the operation related to the temperature control or the like of the thermostatic bath 400 is ended to complete the thermostatic bath control processing, and if not determined, the processing returns to step S703.

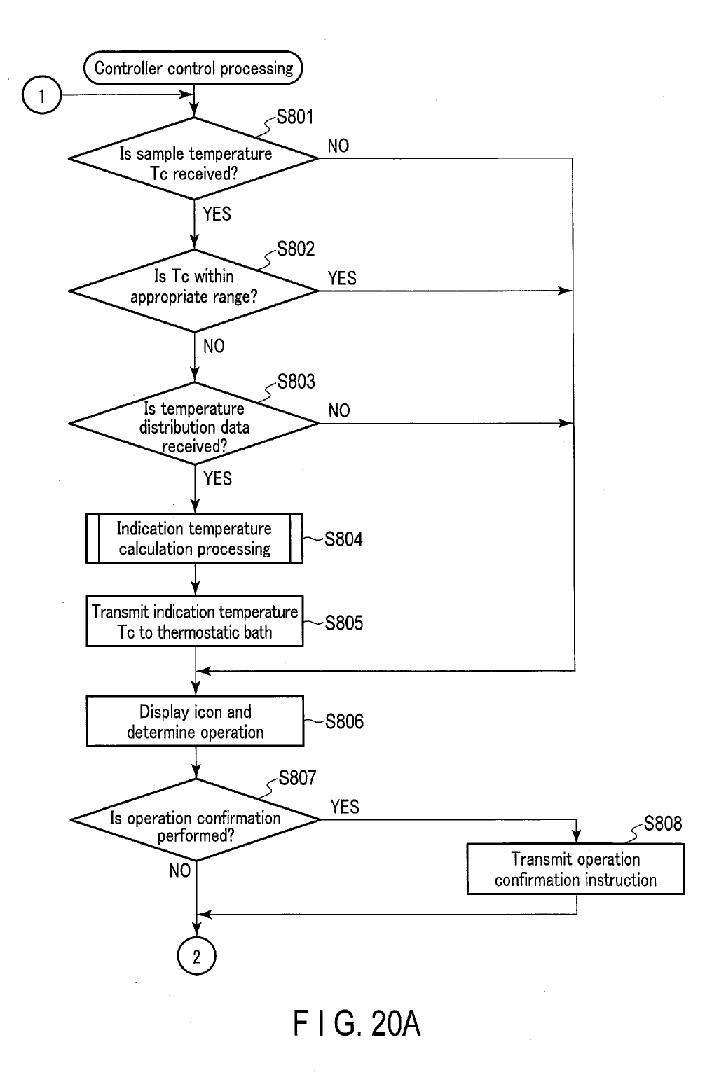

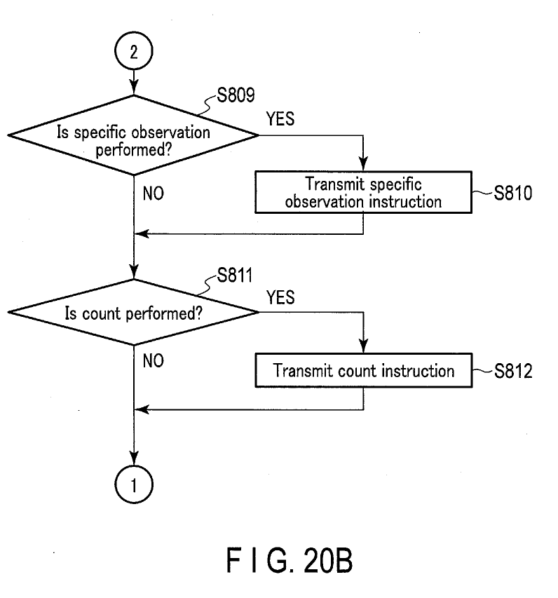

FIGS. 20A and 20B are flowcharts of an example of controller control processing of this embodiment executed by the controller 200, and the operation of the observation system 1 will be explained with reference to FIGS. 20A and 20B. The following description is given through the comparison with the controller control processing according to the first embodiment described above with reference to FIG. 13.

In step S801, the controller side control circuit 210 determines whether or not the sample temperature Tc has been received from the observation apparatus 100. If it is determined that the sample temperature Tc has been received, the processing proceeds to step S802, and if it is not determined, the processing proceeds to step S806.

In step S802, the controller side control circuit 210 determines whether or not the received sample temperature Tc is within the appropriate range in the same manner as in step S133. If it is determined that the received sample temperature Tc is within the appropriate range, the processing proceeds to step S806, and if not determined, the processing proceeds to step S803. In step S803, the control circuit 210 determines whether or not the temperature distribution data 451 has been received from the thermostatic bath 400. If it is determined that the temperature distribution data 451 has been received, the processing proceeds to step S804, and if it is not determined, the processing proceeds to step S806. In step S804, the control circuit 210 executes indication temperature calculation processing. As described above, the indication temperature calculation processing of this embodiment is executed in the controller 200. The indication temperature calculation processing of this embodiment is the same as the indication temperature calculation processing according to the first embodiment executed by the observation apparatus 100 described above with reference to FIG. 10. In step S805, the control circuit 210 transmits the calculated indication temperature Tic to the thermostatic bath 400. The transmitted indication temperature Tic is used as the set temperature To of the thermostatic bath 400.

In steps S806 to S812, the controller side control circuit 210 performs a variety of processing such as operation confirmation, specific observation, or cell count based on the result of the operation determination after icon display in the same manner as in steps S301 to step S307.

As described above, the observation system 1 of this embodiment includes the observation apparatus 100, the controller 200, and the thermostatic bath 400. In the observation system 1, in a state in which the observation apparatus 100 controlled by the controller 200 is placed in the thermostatic chamber 402 of the thermostatic bath 400 together with the sample 300 to be observed, the controller 200 and the observation apparatus 100 perform first communication with each other, the controller 200 and the thermostatic bath 400 perform second communication with each other, and temperature control in the thermostatic chamber 402 is performed based on the set temperature To. The observation apparatus 100 is provided with an imaging unit (imaging section 151), a temperature sensor (sensor section 170), and first control circuitry (observation side control circuit 110). The imaging unit is configured to capture the image of the sample 300. The temperature sensor is configured to measure the internal temperature Te of the observation apparatus 100. The first control circuitry is configured to estimate the sample temperature Tc, which is the temperature of the sample 300, based on the internal temperature Te. The controller 200 is provided with third control circuitry (controller side control circuit 210). The third control circuitry is configured to calculate the indication temperature Tic indicated to the thermostatic bath 400 as the set temperature To, based on the sample temperature Tc acquired from the observation apparatus 100 through the first communication and the temperature distribution data 451 acquired from the thermostatic bath 400 through the second communication. The thermostatic bath 400 is provided with a memory (thermostatic bath side storage circuit 450) and second control circuitry (thermostatic bath side control circuit 410). The memory is configured to store the temperature distribution data 451 of the thermostatic chamber 402. The second control circuitry is configured to perform the temperature control by setting the set temperature To based on the indication temperature Tic acquired from the controller 200 through the second communication.

The thermostatic bath 400 is further provided with a detector 432. The detector 432 is configured to detect information on the control position that is the position of the sample 300 in the thermostatic chamber 402. The first control circuitry calculates the indication temperature Tic based on the information on the control position acquired from the thermostatic bath 400 through the second communication.

That is, in the observation system 1 of this embodiment, the controller 200 calculates the indication temperature Tic based on the current sample temperature Tc estimated by the observation apparatus 100. The thermostatic bath 400 performs temperature control by setting the set temperature To based on the indication temperature Tic calculated by the controller 200. The observation system 1 of this embodiment has advantages similar to those obtained by the observation systems 1 according to the first embodiment and the second embodiment.

### <Modification>

In the observation system 1 according to the first embodiment, in which the communication is performed between the observation apparatus 100 and the controller 200 and between the observation apparatus 100 and the thermostatic bath 400, the calculation of the indication temperature Tic may be performed by the thermostatic bath 400. In this case, in the observation apparatus 100, the sample temperature Tc is estimated based on the internal temperature Te of the observation apparatus 100. The estimated sample temperature Tc is transmitted to the thermostatic bath 400. In the thermostatic bath 400, the indication temperature Tic is calculated based on the received sample temperature Tc and the temperature distribution data 451 stored in the thermostatic bath 400. The temperature control of the thermostatic bath 400 is performed by using the indication temperature Tic calculated by the thermostatic bath 400 as the set temperature To. The thermostatic bath 400 may receive the internal temperature Te from the observation apparatus 100 and estimate the sample temperature Tc.

In the observation system 1 according to the first embodiment, in which the communication is performed between the observation apparatus 100 and the controller 200 and between the observation apparatus 100 and the thermostatic bath 400, the calculation of the indication temperature Tic may be performed by the controller 200. In this case, the sample temperature Tc estimated by the observation apparatus 100 is transmitted to the controller 200. In the controller 200, the indication temperature Tic is calculated based on the sample temperature Tc received from the observation apparatus 100 and the temperature distribution data 451 received from the thermostatic bath 400. The temperature control of the thermostatic bath 400 is performed by using the indication temperature Tic calculated by the controller 200 as the set temperature To. The controller 200 may receive the internal temperature Te of the observation apparatus 100 from the observation apparatus 100 and estimate the sample temperature Tc.

In the observation system 1 according to the second embodiment, in which the communication is performed between the observation apparatus 100 and the controller 200, the observation apparatus 100 may transmit the calculated indication temperature Tic to the controller 200 without displaying the calculated indication temperature Tic on the display of the observation apparatus 100. In this case, the display may not be provided in the observation apparatus 100. Even in such a case, the user can confirm the calculated indication temperature Tic through the controller 200.

In the observation system 1 according to the second embodiment, in which the communication is performed between the observation apparatus 100 and the controller 200, when the thermostatic bath 400 is provided with an imaging device that monitors the inside of the thermostatic chamber 402, the imaging may be performed so as to include the display of the observation apparatus 100, and the acquired image may be displayed on the display 461. Even in such a case, the user can confirm the indication temperature Tic displayed by the observation apparatus 100 while viewing the display 461 of the thermostatic bath 400.

In the observation system 1 according to the second embodiment, in which the communication is performed between the observation apparatus 100 and the controller 200, the calculation of the indication temperature Tic may be performed by the controller 200. For example, the observation apparatus 100 transmits the estimated sample temperature Tc to the controller 200. The controller 200 calculates the indication temperature Tic based on the sample temperature Tc or the like, and displays the calculated indication temperature Tic. The controller 200 may receive the internal temperature Te of the observation apparatus 100 from the observation apparatus 100 and estimate the sample temperature Tc.

In the observation system 1 according to the second embodiment, in which the communication is performed between the observation apparatus 100 and the controller 200, the calculation of the indication temperature Tic may be performed by the thermostatic bath 400. For example, the observation apparatus 100 displays the estimated sample temperature Tc, or transmits and displays the estimated sample temperature Tc to the controller 200. The user inputs the displayed sample temperature Tc to the thermostatic bath 400. The thermostatic bath 400 calculates the indication temperature Tic based on the sample temperature Tc or the like. The thermostatic bath 400 updates the set temperature To of the thermostatic bath 400 based on the calculated indication temperature Tic and performs temperature control. The thermostatic bath 400 may estimate the sample temperature Tc based on the internal temperature Te of the observation apparatus 100 input by the user.

In the observation system 1 according to the third embodiment, in which the communication is performed between the controller 200 and the observation apparatus 100 and between the controller 200 and the thermostatic bath 400, the calculation of the indication temperature Tic may be performed by the observation apparatus 100. For example, the observation apparatus 100 calculates the indication temperature Tic based on the estimated sample temperature Tc and transmits the calculated indication temperature Tic to the controller 200. The thermostatic bath 400 performs temperature control by using the indication temperature Tic received through the controller 200 as the set temperature To.

In the observation system 1 according to the third embodiment, in which the communication is performed between the controller 200 and the observation apparatus 100 and between the controller 200 and the thermostatic bath 400, the controller 200 may receive the internal temperature Te of the observation apparatus 100 from the observation apparatus 100 and estimate the sample temperature Tc.

In the observation system 1 according to the third embodiment, in which the communication is performed between the controller 200 and the observation apparatus 100 and between the controller 200 and the thermostatic bath 400, the calculation of the indication temperature Tic may be performed by the thermostatic bath 400. For example, the thermostatic bath 400 calculates the indication temperature Tic based on the sample temperature Tc received through the controller 200 and performs temperature control by using the calculated indication temperature Tic as the set temperature To. At this time, the observation system 1 can be expressed as follows. The observation system 1 includes the observation apparatus 100 and the thermostatic bath 400. In the observation system 1, in a state in which the observation apparatus 100 is placed in the thermostatic chamber 402 of the thermostatic bath 400 together with the sample 300 to be observed, the observation apparatus 100 and the thermostatic bath 400 communicate with each other and temperature control in the thermostatic chamber 402 is performed based on the set temperature To. The observation apparatus 100 is provided with an imaging unit (imaging section 151), a temperature sensor (sensor section 170), and first control circuitry (observation side control circuit 110). The imaging unit is configured to capture the image of the sample 300. The temperature sensor is configured to measure the internal temperature Te of the observation apparatus 100. The first control circuitry is configured to estimate the sample temperature Tc, which is the temperature of the sample 300, based on the internal temperature Te. The thermostatic bath 400 is provided with a memory (thermostatic bath side storage circuit 450) and second control circuitry (thermostatic bath side control circuit 410). The memory is configured to store the temperature distribution data 451 of the thermostatic chamber 402. The second control circuitry is configured to perform the temperature control by calculating the indication temperature Tic indicated to the thermostatic bath 400 as the set temperature To based on the sample temperature Tc acquired from the observation apparatus 100 through the communication and the temperature distribution data 451, and setting the set temperature To based on the calculated indication temperature Tic. The thermostatic bath 400 may estimate the sample temperature Tc based on the internal temperature Te received from the observation apparatus 100 through the controller 200.

In the observation systems 1 according to the first embodiment and the third embodiment, the information on the control position may be acquired based on the user's input. The user's input may be acquired by the observation apparatus 100 further provided with an input device that acquires a user input, may be acquired by the controller 200, or may be acquired by the thermostatic bath 400. The input device of the observation apparatus 100 is similar to, for example, the input device 274 of the controller 200 and the input device 462 of the thermostatic bath 400. Information on the acquired control position may be transmitted to a device on which the indication temperature calculation processing is executed as appropriate.

In the observation systems 1 according to the first embodiment, the second embodiment, and the third embodiment, the control position may be a predetermined position. For example, a marker, a guide, or the like indicating the position at which the observation apparatus 100 has to be placed may be provided in each shelf 403 so that the observation apparatus 100 is placed at an appropriate position. The information on the control position may be acquired by image recognition. For example, the detector 432 of the thermostatic bath 400 may be provided with an imaging device for monitoring the inside of the thermostatic bath 400 and an image processing circuit. In this case, the detector 432 analyzes the image acquired by the imaging device by using the image processing circuit, and detects the position at which the observation apparatus 100 is placed by image recognition. The detected control position may be transmitted to the observation apparatus 100 or the controller 200 through communication, or may be displayed on the display 461 of the thermostatic bath 400. Such image recognition may be performed by the observation apparatus 100. In this case, for example, the information on the control position may be included in the imaging range of the observation apparatus 100, such as the rear surface of the shelf 403 of the thermostatic bath 400. The information on the control position may be text information, a bar code, a two-dimensional code, or the like.

In the observation systems 1 according to the first embodiment, the second embodiment, and the third embodiment, the information on the control position is not limited to the height (Z direction), but the position in the X direction or the Y direction may be further included. For example, a plurality of detectors 432 may be placed on each shelf 403. In this case, for example, the detector 432 can further detect at which position (X position and Y position) of the upper shelf 403a (lower shelf 403b) the observation apparatus 100 is placed, and whether or not the observation apparatus 100 is placed at an appropriate position, and the like.

In the observation systems 1 according to the first embodiment, the second embodiment, and the third embodiment, for example, the temperature distribution data 451 is not limited to each type of the thermostatic bath 400, but may be set for each set temperature To of the thermostatic bath 400 and may be set (registered) for each of the X position and the Y position in the thermostatic bath 400, such as the center of the shelf 403, near the wall, or the like. In this case, since the temperature control is performed based on the temperature distribution data 451 corresponding to the situation or position at which the observation apparatus 100 is placed, more appropriate temperature control can be realized.

In the observation systems 1 according to the first embodiment and the third embodiment, the memory that stores the temperature distribution data 451 is not limited to the thermostatic bath side storage circuit 450, and may be the observation side storage circuit 130 or the controller side storage circuit 230. In the observation system 1 according to the second embodiment, the memory that stores the temperature distribution data 451 may be the observation side storage circuit 130 or the controller side storage circuit 230. The temperature distribution data 451 may be previously stored, may be acquired from an external memory, or may be acquired from a server or the like on the Internet.

In the observation systems 1 according to the first embodiment, the second embodiment, and the third embodiment, a plurality of observation apparatuses 100 may be placed in the thermostatic chamber 402. In this case, the set temperature To of the thermostatic bath 400 may be calculated based on a plurality of indication temperatures Tic. The set temperature To may be the average value of all the indication temperatures Tic, or may be the average value of the maximum value and the minimum value of the plurality of indication temperatures Tic. In addition, there may be a specification in which priorities are assigned to the plurality of observation apparatuses 100, and the indication temperature Tic corresponding to the observation apparatus 100 having the highest priority or the weighted average value corresponding to the priority is used as the set temperature To. The plurality of observation apparatuses 100 may be placed on one shelf. In this case, in the calculation of the indication temperature Tic, the same temperature distribution data 451 may be used, or the temperature distribution data 451 corresponding to each of the positions may be respectively used.

In the observation systems 1 according to the first embodiment, the second embodiment, and the third embodiment, information related to heat generation inside the observation apparatus 100 such as, for example, heat generation of the imaging device due to repeated imaging, may be taken into consideration when the sample temperature Tc is estimated or the indication temperature Tic is calculated. In this case, the temperature rise of the sample 300 caused by heat generation inside the observation apparatus 100 can also be suppressed, and more appropriate temperature control can be realized.

Since the inside of the observation apparatus 100 is hermetically sealed, when a pressure difference occurs between the inside and outside of the observation apparatus 100 due to internal heat generation or a change in external environment, deformation may occur in the transparent plate 102 or the like of the observation apparatus 100, resulting in poor observation or destruction of the observation apparatus 100. In the observation systems 1 according to the first embodiment, the second embodiment, and the third embodiment, a pressure valve or the like may be provided in the observation apparatus 100 so as to suppress such an increase in internal pressure.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

In the embodiments, a part named as a section or a unit may be structured by a dedicated circuit or a combination of a plurality of general purpose circuits, and may be structured by a combination of a microcomputer operable in accordance with a pre-programmed software, a processor such as a CPU, or a sequencer such as an FPGA. In addition, a design where a part of or total control is performed by an external device can be adopted. In this case, a communication circuit is connected by wiring or wirelessly. Communication may be performed by means of Bluetooth, Wi-Fi, a telephone line, or a USB. A dedicated circuit, a general purpose circuit, or a controller may be integrally structured as an ASIC. A specific mechanical functionality (that can be substituted by a robot when a user images while moving) may be structured by various actuators and mobile concatenating mechanisms depending on the need, and may be structured by an actuator operable by a driver circuit. The driver circuit is controlled by a microcomputer or an ASIC in accordance with a specific program. The control may be corrected or adjusted in detail in accordance with information output by various sensors or peripheral circuits.

What is claimed is:

1. An observation system, in a state in which at least one observation apparatus is placed in a thermostatic chamber of a thermostatic bath together with a sample to be observed, in which the observation apparatus and the thermostatic bath communicate with each other and temperature control in the thermostatic chamber is performed based on a set temperature, the observation system comprising:
the observation apparatus comprising:
an imaging unit configured to capture an image of the sample;
a temperature sensor configured to measure an internal temperature of the observation apparatus; and
first control circuitry configured to estimate a sample temperature, which is a temperature of the sample, based on the internal temperature and calculate an indication temperature indicated to the thermostatic bath as the set temperature based on the sample temperature and temperature distribution data acquired from the thermostatic bath through the communication; and
the thermostatic bath comprising:
a memory configured to store the temperature distribution data of the thermostatic chamber; and
second control circuitry configured to perform the temperature control by setting the set temperature based on the indication temperature acquired from the observation apparatus through the communication.

2. The observation system according to claim 1, wherein the thermostatic bath further comprises detection circuitry configured to detect information on a control position that is a position of the sample in the thermostatic chamber, and
the first control circuitry calculates the indication temperature based on the information on the control position acquired from the thermostatic bath through the communication.

3. The observation system according to claim 2, wherein the sample is placed on the observation apparatus, and
the control position is a position of the observation apparatus in the thermostatic chamber.

4. The observation system according to claim 1, wherein the observation apparatus includes a first observation apparatus and a second observation apparatus, and
when the first observation apparatus and the second observation apparatus are placed in the thermostatic chamber, the second control circuitry sets the set temperature based on a first indication temperature and a second indication temperature respectively corresponding to the first observation apparatus and the second observation apparatus, and the indication temperature includes the first indication temperature and the second indication temperature.

5. The observation system according to claim 1, wherein the temperature distribution data indicating a relationship between positions and temperatures in the thermostatic chamber.

6. The observation system according to claim 5, wherein the temperature distribution data includes temperature distributions corresponding to the set temperatures of the thermostatic bath or the positions in the thermostatic chamber.

7. The observation system according to claim 1, wherein the temperature control is a control that is performed based on a difference between the set temperature and an internal temperature of the thermostatic chamber measured at a reference control position, and
the first control circuitry:
calculates a current temperature distribution in the thermostatic chamber by using the temperature distribution data, a control position that is a position of the observation apparatus in the thermostatic chamber, and the sample temperature acquired as a temperature of the control position;
calculates the reference control position by using the current temperature distribution and the current set temperature;
calculates a target temperature distribution by using the current temperature distribution and a target temperature of the control position; and
calculates a target temperature of the reference control position as the indication temperature by using the reference control position and the target temperature distribution.

8. An observation apparatus provided with an imaging unit configured to capture an image of a sample to be observed, the observation apparatus comprising:
a temperature sensor configured to measure an internal temperature of the observation apparatus;
a memory configured to store temperature distribution data of a thermostatic chamber of a thermostatic bath, in which temperature control is performed based on a set temperature in the thermostatic chamber in which the observation apparatus is placed together with the sample; and
control circuitry configured to estimate a sample temperature, which is a temperature of the sample, based on the internal temperature, calculate an indication temperature recommended as the set temperature of the thermostatic bath based on the sample temperature and the temperature distribution data, and generate display information for displaying the indication temperature.

9. The observation apparatus according to claim 8, further comprising an input interface configured to acquire information on a control position, which is a position of the sample in the thermostatic chamber, based on a user's input,
wherein the control circuitry calculates the indication temperature based on the information on the control position.

10. The observation apparatus according to claim 9, wherein the sample is placed on the observation apparatus, and
the control position is a position of the observation apparatus in the thermostatic chamber.

11. The observation apparatus according to claim 8, further comprising an input interface configured to acquire a user's input,
wherein the temperature distribution data includes temperature distributions corresponding to the set temperatures of the thermostatic bath or positions in the thermostatic chamber, and
the control circuitry selects the temperature distribution to be used for the calculation of the indication temperature based on an output of the input interface.

12. The observation apparatus according to claim 8, further comprising communication circuitry configured to transmit the display information for displaying the indication temperature to the outside.

13. The observation apparatus according to claim 8, wherein the temperature control is a control that is performed based on a difference between the set temperature and an internal temperature of the thermostatic chamber measured at a reference control position, and
wherein the control circuitry:
calculates a current temperature distribution in the thermostatic chamber by using the temperature distribution data, a control position that is a position of the observation apparatus in the thermostatic chamber, and the sample temperature acquired as a temperature of the control position;
calculates the reference control position by using the current temperature distribution and the current set temperature;
calculates a target temperature distribution by using the current temperature distribution and a target temperature of the control position; and
calculates a target temperature of the reference control position as the indication temperature by using the reference control position and the target temperature distribution.

14. A temperature control method, which is performed in a state in which an observation apparatus is placed together with a sample in a thermostatic chamber of a thermostatic bath in which temperature control is performed based on a set temperature, the observation apparatus including an imaging unit configured to capture an image of the sample, the temperature control method comprising:
registering temperature distribution data of the thermostatic chamber;
measuring an internal temperature of the observation apparatus;
estimating a sample temperature, which is a temperature of the sample, based on the internal temperature;
calculating an indication temperature indicated to the thermostatic bath as the set temperature based on the sample temperature and the temperature distribution data; and
setting the set temperature based on the indication temperature.

15. The temperature control method according to claim 14, wherein the temperature distribution data indicating a relationship between positions and temperatures in the thermostatic chamber.

16. The temperature control method according to claim 14, wherein the temperature control is a control that is performed based on a difference between the set temperature and an internal temperature of the thermostatic chamber measured at a reference control position, and
the calculating of the indication temperature includes:
calculating a current temperature distribution in the thermostatic chamber by using the temperature distribution data, a control position that is a position of the observation apparatus in the thermostatic chamber, and the sample temperature acquired as a temperature of the control position;
calculating the reference control position by using the current temperature distribution and the current set temperature;
calculating a target temperature distribution by using the current temperature distribution and a target temperature of the control position; and
calculating a target temperature of the reference control position as the indication temperature by using the reference control position and the target temperature distribution.

17. The temperature control method according to claim 16, wherein the sample is placed on the observation apparatus, and
the control position is a position of the observation apparatus in the thermostatic chamber.

18. The temperature control method according to claim 14, wherein, when the observation apparatuses are placed in the thermostatic chamber, the setting of the set temperature is performed based on the indication temperatures calculated for each of the observation apparatuses.
